(19) United States
(12) Patent Application Publication
(10) Pub. No.: US 2024/0252490 A1
(43) Pub. Date: Aug. 1, 2024

(54) Combination Therapies Comprising Kras Inhibitors and SPH2 Inhibitors

(71) Applicant: HUYABIO International, LLC, San Diego, CA (US)

(72) Inventors: Farbod SHOJAEI, San Diego, CA (US); Mireille GILLINGS, San Diego, CA (US)

(21) Appl. No.: 18/517,019

(22) Filed: Nov. 22, 2023

Related U.S. Application Data
(60) Provisional application No. 63/427,731, filed on Nov. 23, 2022.

(51) Int. Cl. A61K 31/4985; A61K 31/519; A61P 35/04

(52) U.S. Cl. CPC A61K 31/4985; A61K 31/519; A61P 35/04

(57) ABSTRACT

Provided herein are combinations that include a KRAS inhibitor and a SPH2 inhibitor and methods of treating cancer.

### CROSS-REFERENCE

This application claims the benefit of priority from U.S. Provisional Application 63/427,731, filed Nov. 23, 2022, which is incorporated herein by reference in its entirety.

### FIELD OF THE DISCLOSURE

The present disclosure relates to combinations comprising a SHP2 inhibitor and a KRAS inhibitor. Such combinations include therapeutic methods of using a SHP2 inhibitor in combination with a KRAS inhibitor and the use of such combinations in the treatment of cancer.

### BACKGROUND OF THE DISCLOSURE

SH2-containing protein tyrosine phosphatase 2 (SHP2) belongs to the protein tyrosine phosphatase family, which is involved in regulating cell proliferation, survival, differentiation, migration and apoptosis. SHP2 can regulate Ras-mitogen-activated protein kinase, Janus kinase-signal transducer and activator of transcription (JAK-STAT) or phosphoinositide 3-kinase-AKT and nuclear factor κB (NF-κB) and other signaling pathways. SHP2 is also the main regulator of the immune checkpoint signaling pathway of programmed cell death protein-1 (PD-1) and B and T lymphocyte attenuation factor (BTLA), which may be related to tumor immunosuppression. Therefore, activation of SHP2 has become a feasible anti-tumor strategy. In addition, SHP2 mutations rarely occur in tumors, thus making it an achievable target for cancer therapy.

In comparison to SHP2, the RAS genes, which include H-, N-, and K-RAS variants, comprise the most frequently mutated family of oncogenes in cancer. KRAS is the Kirsten rat sarcoma viral oncogene homologue. Mutations in KRAS—such as the G12C mutation—are found in most pancreatic cancers, half of colorectal cancers and a third of lung cancer cases and is thus responsible for a substantial proportion of cancer deaths. Thus, compounds that inhibit KRAS activity are prospective candidates for anti-tumor strategy.

Notwithstanding years of discovery efforts to develop effective anti-tumor strategies, single agent therapy still fails to meet the need for effective anti-tumor strategy in many cancers. Moreover, significant numbers of tumors are either resistant, or become refractory, to single agents.

Accordingly, there is a need for new therapies, including, for example, combination therapies for the treatment of cancers. Provided herein are combinations comprising SHP2 and KRAS inhibitors, including uses and methods of treating cancer comprising those combinations.

### BRIEF SUMMARY

Provided herein, inter alia, are methods of treating and/or preventing cancer comprising a KRAS inhibitor and a SHP2 inhibitor.

In one embodiment, provided herein is a method of treating or preventing brain metastasis comprising administration of a KRAS inhibitor compound of Formula I, or a pharmaceutically acceptable salt or solvate thereof:

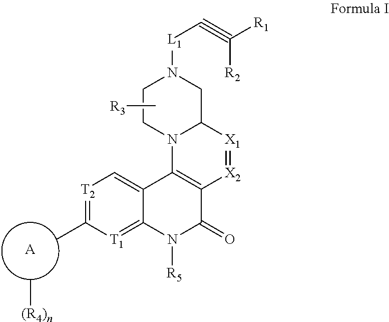

wherein the variables R₁, R₂, R₃, R₄, R₅, L₁, X₁, X₂, T₁, T₂, A, and n are set forth herein.

In some embodiments, treating brain metastasis comprises suppression of growth of a brain tumor and the effective amount of the compound is an amount effective to suppress growth of a brain tumor.

In some embodiments, the brain tumor is a secondary tumor. Whereas, in some other embodiments, the primary tumor is not a brain tumor. In some embodiments, the compound of Formula I penetrates the blood-brain barrier (BBB).

In some embodiments, the KRAS inhibitor compound of Formula I is Formula Ia (HBI-2438):

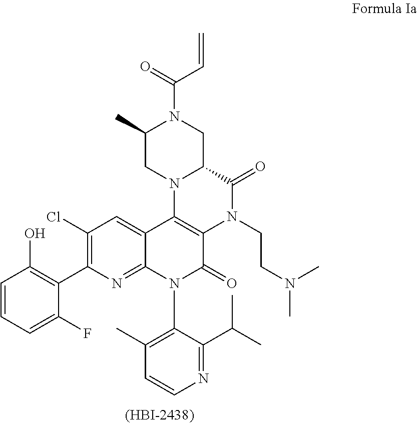 [SMILES: C=CC(=O)N1C[C@@H]2C(=O)N(CCN(C)C)c3c(c4cc(Cl)c(-c5c(O)cccc5F)nc4n(-c4c(C)ccnc4C(C)C)c3=O)N2C[C@H]1C]

or an enantiomer or a pharmaceutically acceptable salt thereof.

In some embodiments, the compound of Formula I is administered to the subject in an amount of about 1 to about 100 mg/kg per day for at least one day.

In some embodiments, the method further comprises administering an effective amount a compound of Formula II, wherein the compound of Formula II is selected from compounds of the formula:

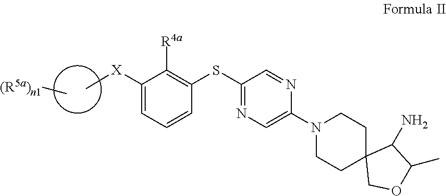

or a pharmaceutically acceptable salt thereof, wherein the variables n1, X, R^{4a} and R^{5a} are set forth herein.

In some embodiments, the amount of the compound of Formula II is synergistic with the amount of the compound of Formula I.

In some embodiments, the amount of the compound of Formula II is synergistic with the amount of the compound of Formula I to treat brain metastasis. In some other embodiments, the combined amounts of the compound of Formula I and Formula II are effective to suppress growth or induce regression of a brain tumor.

In some embodiments, the brain tumor is secondary tumor when the compound of Formula Ia and the compound of Formula IIa are used together.

In some embodiments, the primary tumor is not a brain tumor when the compound of Formula Ia and the compound of Formula IIa are used together.

In some embodiments, the brain tumor is secondary when the compound of Formula Ia and the compound of Formula IIa are used together.

In some embodiments, the compound of Formula II is Formula IIa (HBI-2376):

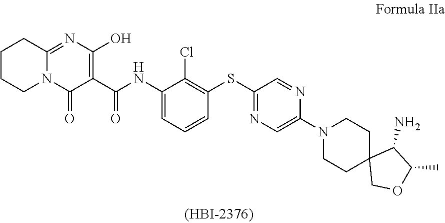 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

or a racemate or a pharmaceutically acceptable salt thereof.

In some embodiments, the compound of Formula I is administered to the subject in an amount of about 0.5 to about 100 mg/kg per day for at least one day. In some other embodiments, the compound of Formula II is administered to the subject in an amount of about 5 to about 25 mg/kg per day. In some embodiments, the compound of Formula I is a compound of Formula Ia and the compound of Formula II is a compound of Formula IIa.

In some embodiments, provided herein is a method of treating or preventing brain metastasis comprising administration of a KRAS inhibitor compound and a SPH2 inhibitor compound, or a pharmaceutically acceptable salts or solvates thereof.

In some embodiments, each of the KRAS inhibitor compound and the SPH2 inhibitor compound is a small molecule compound.

In some embodiments, described herein are combinations (e.g., combination therapies, such as therapeutic methods and uses, kits and compositions) for treating diseases comprising cancer.

Some embodiments of the present disclosure comprise a first pharmaceutical composition and a second pharmaceutical composition. The first pharmaceutical composition comprises the compound of Formula I and the second pharmaceutical comprises the compound of Formula II. In some embodiments, each of the first pharmaceutical composition and the second pharmaceutical composition is administered via the same routes of administration. In some embodiments, each of the first pharmaceutical composition and the second pharmaceutical composition is administered via the different routes of administration.

In some embodiments, provided herein is a pharmaceutical composition comprising a first pharmaceutical composition comprising a KRAS inhibitor compound and a second pharmaceutical composition comprising a SPH2 inhibitor compound, and a pharmaceutically acceptable excipient.

In some embodiments, described herein are combinations (e.g., combination therapies, such as therapeutic methods and uses, kits and compositions) for treating cancer. In some embodiments, the kit comprises a combination of any of one of the embodiments described herein or a pharmaceutical composition of the embodiments described herein. In some embodiments, the kit further comprises at least one administration device. In some embodiments, one or more components of the kit are sterilized. In some embodiments, the combinations described herein include a KRAS inhibitor and a SPH2 inhibitor. In some embodiments, the combinations described herein include a KRAS inhibitor, a SPH2 inhibitor and a third anti-cancer agent.

In some embodiments, provided herein is a method of treating cancer in a subject in need thereof, comprising administering to the patient a combination comprising a therapeutically effective amount of a compound of Formula Ia, or a pharmaceutically acceptable salt or solvate thereof:

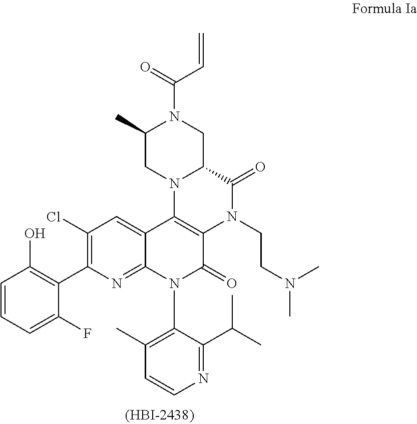 [SMILES: C=CC(=O)N1C[C@@H]2C(=O)N(CCN(C)C)c3c(c4cc(Cl)c(-c5c(O)cccc5F)nc4n(-c4c(C)ccnc4C(C)C)c3=O)N2C[C@H]1C]

and a therapeutically acceptable amount of a SPH2 inhibitor.

In some embodiments, provided herein is a method of treating cancer in a subject in need thereof, comprising administering to the patient a combination comprising a therapeutically effective amount of a KRAS inhibitor and a SPH2 inhibitor of Formula IIa (HBI-2376):

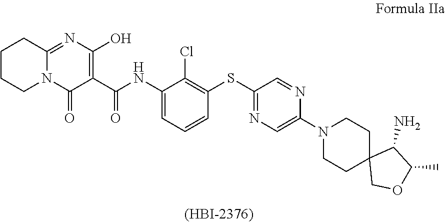 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

or a pharmaceutically acceptable salt or solvate thereof.

In some embodiments, the method comprises administering the compound of Formula I, or a pharmaceutically acceptable salt or solvate thereof; and the compound of Formula II simultaneously or sequentially. In some embodiments, the method comprises administering the compound of Formula I, or a pharmaceutically acceptable salt or solvate thereof; and the compound of Formula II as a regimen.

In some embodiments, the patient is treatment naïve. In some other embodiments, the patient is treatment naïve for brain tumor. In some embodiments, the method comprises administering the compound of Formula I and the compound of Formula II or pharmaceutically acceptable salts or solvates thereof; to said patient as a first line therapy. In some embodiments, the method comprises administering the compound of Formula I and the compound of Formula II or pharmaceutically acceptable salts or solvates thereof; to said patient as a second, third, fourth, fifth, or sixth line of treatment.

In some embodiments, the method comprises administering the compound of Formula I and the compound of Formula II, or a pharmaceutically acceptable salt or solvate thereof, to a patient following treatment with at least one other anti-cancer therapy, wherein the anti-cancer therapy is chemotherapy, radiotherapy, surgery, targeted therapy, immunotherapy, or a combination of two or more thereof.

In some embodiments, the method comprises inhibiting metastasis of the cancer in a patient in need of such treatment. In some other embodiments, the method comprises inhibiting brain metastasis in a patient in need of such treatment. In some embodiments, the method of treating cancer prolongs the time to disease progression of cancer in the patient. In some embodiments, the method of treating cancer prolongs the survival of the patient. In some embodiments, the method of treating cancer increases progression-free survival of the patient. In some embodiments, the method of treating cancer reduces a tumor or tumor burden in the patient. In some embodiments, the method reduces or prevents metastasis of a primary tumor in the patient in need of treatment.

Other objects, features and advantages of the combinations and methods described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1A is a graph illustrating the effect of compound of Formula I (HBI-2438) on tumor volume in mice bearing H1373-Luc+ICA tumors. These results are summarized in Table 1 in Example 1.

FIG. 1B is a graph depicting the effect of compound of Formula I (HBI-2438) on body weight in mice bearing H1373-Luc+ICA tumors.

FIG. 2A is a graph showing the synergistic effect of compound of Formula I (HBI-2438) and compound of Formula II (HBI-2376) on tumor volume in mice bearing H1373-Luc+ICA tumors.

FIG. 2B is a graph illustrating the synergistic effect of compound of Formula I and compound of Formula II on tumor volume in mice bearing H1373-Luc+ICA tumors. The data depicted in FIG. 2B is a subset of the FIG. 2A data, representing only the combination therapy at various doses of HBI-2376 (SHP2 inhibitor) and HBI-2438 (KRAS inhibitor).

FIG. 2C is a graph showing the synergistic effect of compound of Formula I (HBI-2438) and compound of Formula II (HBI-2376) on body weight in mice bearing H1373-Luc+ICA tumors.

FIG. 3A is a graph illustrating the relative expression of ERK, pERK, DUSP6 in different treatment groups, wherein G1 represents vehicle, G2 represents 5 mg/kg HBI-2376 (SHP2 inhibitor), G5-G7 represent 10, 30, and 100 mg/kg, respectively, of HBI-2438 (KRAS inhibitor), and G3 and G4 represent positive controls.

FIG. 3B is a graph of the relative expression of ERK and pERK in the treatment groups referenced in FIG. 3A.

FIG. 3C is a graph of the relative expression of DUSP6 in the treatment groups referenced in FIG. 3A.

FIG. 4A shows the results of western blot analysis representing the relative expression of ERK, pERK, DUSP6 in different treatment groups, wherein HBI-2376 is a SHP2 inhibitor of the invention described herein and HBI-2438 is a KRAS inhibitor of the invention described herein.

FIG. 4B is a graph representing the relative expression of ERK and pERK in different treatment groups, wherein HBI-2376 is a SHP2 inhibitor of the invention described herein and HBI-2438 is a KRAS inhibitor of the invention described herein.

FIG. 4C is a bar graph representing the relative expression of DUSP6 in different treatment groups, wherein HBI-2376 is a SHP2 inhibitor of the invention described herein and HBI-2438 is a KRAS inhibitor of the invention described herein.

FIG. 5A shows the results of western blot analysis representing the relative expression of ERK, pERK, DUSP6 in different treatment groups, wherein HBI-2376 is a SHP2 inhibitor of the invention described herein and HBI-2438 is a KRAS inhibitor of the invention described herein.

FIG. 5B is a graph representing the relative expression of ERK and pERK in different treatment groups, wherein HBI-2376 is a SHP2 inhibitor of the invention described herein and HBI-2438 is a KRAS inhibitor of the invention described herein.

FIG. 5C is a graph representing the relative expression of DUSP6 in different treatment groups, wherein HBI-2376 is a SHP2 inhibitor of the invention described herein and HBI-2438 is a KRAS inhibitor of the invention described herein.

FIG. 6A is a graph depicting the relative expression of DUSP6 in different treatment groups, wherein HBI-2376 is a SHP2 inhibitor of the invention described herein and HBI-2438 is a KRAS inhibitor of the invention described herein.

FIG. 6B is a graph showing the relative gene expression of DUSP6 in the presence of the vehicle alone, vehicle+HBI-2376 (SHP2 inhibitor)+HBI-2438 (KRAS inhibitor) combination treatment as disclosed herein in comparison with the combination treatment with positive comparator compounds.

### DETAILED DESCRIPTION

### Definitions

All patents, applications, published applications and other publications cited herein are incorporated by reference in their entirety. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

The term “effective amount” refers to the amount of a therapy (e.g., used in a method provided herein) which is sufficient to accomplish a stated purpose or otherwise achieve the effect for which it is administered. An “effective amount” can be sufficient to reduce and/or ameliorate the progression, development, recurrence, severity and/or duration of a given disease, disorder or condition and/or a symptom related thereto. An “effective amount” can be a “therapeutically effective amount” which refers to an amount sufficient to provide a therapeutic benefit such as, for example, the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. A “therapeutically effective amount” of a compound used in a method described herein can enhance the therapeutic efficacy of another therapeutic agent.

The term “regimen” refers to a protocol for dosing and timing the administration of one or more therapies (e.g., methods described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in some instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In some instances the term refers to active agents such as an anti-cancer agent described herein. The terms “therapy” and “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

The term “patient” or “subject” refers to a mammal, such as a human, bovine, rat, mouse, dog, monkey, ape, goat, sheep, cow, or deer. In some embodiments, a patient as described herein is human.

The terms “inhibition,” “inhibit,” “inhibiting” refer to a reduction in the activity, binding, or expression of a polypeptide or reduction or amelioration of a disease, disorder, or condition or a symptom thereof. “Inhibiting” as used here can include partially or totally blocking stimulation, decreasing, preventing, or delaying activation or binding, or inactivating, desensitizing, or down-regulating protein or enzyme activity or binding. In in vivo contexts, “inhibit” and its variants may include “treat” and its variants.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The terms “treat”, “treating” or “treatment” refer to administration of a therapy to a subject or patient that may be evaluated by any indicia of success or amelioration of the progression, severity, and/or duration of a disease, pathology or condition, including any objective or subjective parameter, such as abatement, remission, diminishing of symptoms, or making the injury, pathology or condition more tolerable to the patient, slowing the rate of degeneration or decline, making the final point of degeneration less debilitating, or improving a subject's or patient's physical or mental well-being.

The term “enhance” refers to an increase or improvement in the function or activity of a first compound after administering to a subject or patient, or contacting a tissue, organ or cell with, a combination comprising the first compound and a second compound, wherein such increase or improvement is measured in comparison to administration or contact with the first or second compound alone.

The term “administering” refers to the act of delivering a combination or composition described herein into a subject by an acceptable route of administration. Such routes may include oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset or diagnosis of the disease, disorder, or condition, or its symptoms but, in some instances, can occur before the onset of the disease, disorder, or condition, or its symptoms (e.g., prophylactic administration for patients prone to such a disease, disorder, or condition).

The term “coadministration” refers to administration of two or more agents (e.g., a combination described herein and another active agent such as an anti-cancer agent described herein). The timing of coadministration depends in part on the combination and compositions administered, and can include administration of members of the combination at the same time, just prior to, or just after the administration of one or more additional therapies. For example cancer therapies, such as chemotherapy, hormonal therapy, radiotherapy, or immunotherapy, may be administered on the same day or on different days, taking into account the sensitivity of the patient, toxicity of the administered agents, etc. The compound of the invention can be administered alone or can be coadministered to the patient. Coadministration is meant to include simultaneous or sequential administration of the compound individually or in combination (more than one compound or agent in a single formulation). Thus, the preparations can also be combined, when desired, with other active substances (e.g., to reduce metabolic degradation). The compounds described herein can be used in combination with one another and with other active agents known to be useful in treating cancer.

The term “anti-cancer agent” is used in accordance with its plain ordinary meaning and refers to a composition having anti-neoplastic properties or the ability to inhibit the growth or proliferation of cells of one or more types of cancer. In some embodiments, an anti-cancer agent is a chemotherapeutic agent. In some embodiments, an anti-cancer agent is an agent identified herein having utility in methods of treating cancer. In some embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” refers to a therapy or regimen that includes administration of a chemotherapeutic or anti-cancer agent described herein.

“Pharmaceutically acceptable” as used herein, refers a material, such as a carrier or diluent, which does not significantly interfere with the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a salt formulation of a compound that does not cause significant irritation to an organism to which it is administered and does not significantly interfere with the biological activity and properties of the compound. In some embodiments, pharmaceutically acceptable salts are obtained by reacting a compound disclosed herein with an acid or base, as the case may be, to form a salt.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

As used herein “metastatic brain tumor” or “metastatic brain cancer” indicates cancer in the brain arising from a separate, metastasized primary tumor. In some embodiments, the primary tumor originates outside the blood brain barrier and metastasizes to form one or more metastatic colonies inside the blood brain barrier. In some embodiments, “inside the blood brain barrier” includes within the lumen of the carotid artery.

The chemistry terms below, as used herein, have the following meanings, unless indicated otherwise:

“Oxo” refers to the ═O substituent.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. Preferably, the C₁-C₁₀ alkyl is any one of methyl, ethyl, n-propyl, isopropyl, and tert-butyl. Unless stated otherwise specifically in the specification, an alkyl group may be optionally substituted as described below.

“Alkylene” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy. The term “C₁-C₁₀ alkoxy” alone or in combination means the group C₁-C₁₀ alkyl-O—, wherein “C₁-C₁₀ alkyl” means as defined above, which includes, but not limited to, methoxy (—OCH₃), ethoxy (—OCH₂CH₃), n-propoxy (—OCH₂CH₂CH₃), iso-propoxy (—OCH(CH₃)₂), n-butoxy (—OCH₂CH₂CH₂CH₃), sec-butoxy (—OCH(CH₃)CH₂CH₃), iso-butoxy (—OCH₂CH(CH₃)₂), tert-butoxy (—OC(CH₃)₃), etc.

“Heteroalkyl” refers to an alkyl radical as described above where one or more carbon atoms of the alkyl is replaced with a O, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized π-electron system containing 4n+2π electrons, where n is an integer. Aromatics can be optionally substituted. The term “aromatic” includes both aryl groups (e.g., phenyl, naphthyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

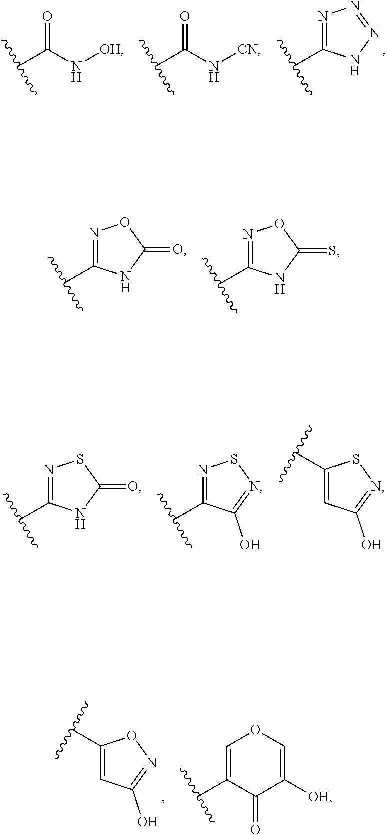

and the like.

“Cycloalkyl” refers to a monocyclic or polycyclic non-aromatic radical, wherein each of the atoms forming the ring (i.e., skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. In some embodiments, a cycloalkyl is a C₃-C₆cycloalkyl. In some embodiments, the cycloalkyl is monocyclic, bicyclic or polycyclic. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, decalinyl and adamantyl. In some embodiments, the cycloalkyl is monocyclic. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the cycloalkyl is bicyclic. Bicyclic cycloalkyl groups include fused bicyclic cycloalkyl groups, spiro bicyclic cycloalkyl groups, and bridged bicyclic cycloalkyl groups. In some embodiments, cycloalkyl groups are selected from among spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, 3,4-dihydronaphthalen-1(2H)-one and decalinyl. In some embodiments, the cycloalkyl is polycyclic. Polycyclic radicals include, for example, adamantyl, and. In some embodiments, the polycyclic cycloalkyl is adamantyl. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, bicyclic ring (which may include a fused bicyclic heterocycloalkyl (when fused with an aryl or a heteroaryl ring, the heterocycloalkyl is bonded through a non-aromatic ring atom), bridged heterocycloalkyl or spiro-heterocycloalkyl), or polycyclic. In some embodiments, the heterocycloalkyl is monocyclic or bicyclic. In some embodiments, the heterocycloalkyl is monocyclic. In some embodiments, the heterocycloalkyl is bicyclic. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted. In some embodiments, the term “3-12 membered heterocyclic group” refers to a saturated or partially unsaturated monocyclic ring containing 3-12, particularly 5-12, more particularly 5-7 carbon atoms and heteroatoms or heteroatom groups or a polycyclic heterocyclic group, the heteroatom or heteroatom group is selected from N, NH, O, C(O), S(O)ₘ (where m is 0, 1 or 2). In some embodiments, the 3-12-membered heterocyclic groups include aziridinyl, azetidinyl, oxetanyl, pyrrolidinyl, tetrahydrofuranyl, tetrahydrothienyl, piperidinyl, morpholinyl, piperazinyl, thiomorpholine, tetrahydropyranyl, 1,1-dioxothiomorpholinyl, butyrolactamyl, valerolactam, caprolactam, butyrolactone, valerolactone, or caprolactone.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

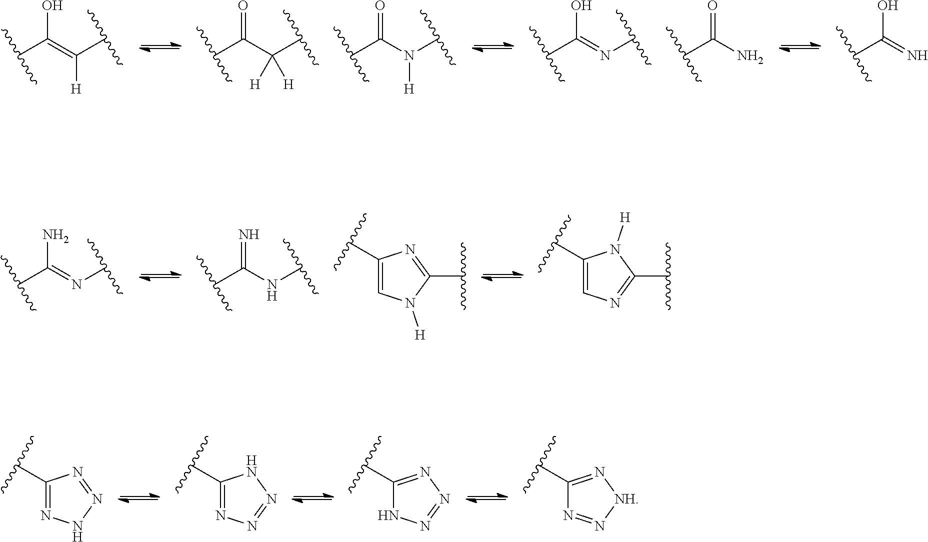

### Combinations

In some embodiments, described herein are combinations (e.g., combination therapies, such as therapeutic methods and uses, kits and compositions) for treating cancer. In some embodiments, the combinations described herein comprise a KRAS inhibitor and a SPH2 inhibitor. In some embodiments, a combination may comprise a first pharmaceutical composition and a second pharmaceutical composition. In some embodiments, the first pharmaceutical composition comprises a KRAS inhibitor compound of Formula I (e.g., Formula Ia (HBI-2438)) and the second pharmaceutical composition comprises a SPH2 inhibitor compound of Formula II (e.g., Formula IIa (HBI-2376)). In some embodiments, the first pharmaceutical composition and the second pharmaceutical composition are co-packaged as a kit, which may further include instructions for co-administration of the first and second pharmaceutical compositions. In some embodiments, the first and second compositions may be packaged separately for combination in a clinical setting by administering them to a patient within a time frame during which the patient derives clinical benefit from the first pharmaceutical composition and the second pharmaceutical composition at the same time. In some embodiments, a combination comprises a unit dosage form of a pharmaceutical composition comprising a KRAS inhibitor and a SPH2 inhibitor. In some embodiments, a combination comprises a first pharmaceutical composition comprising a KRAS inhibitor for use in the treatment of cancer in combination with a second pharmaceutical composition comprising a SPH2 inhibitor. In some embodiments, a combination comprises a use of KRAS inhibitor for preparation of a first pharmaceutical composition for use in the treatment of cancer in combination with a second pharmaceutical composition comprising a SPH2 inhibitor.

In some embodiments, the KRAS inhibitor is a fused pyridine compound, e.g. of Formula I, or Formula Ia, as described herein.

In some embodiments, the SHP2 inhibitor is a pyrazine derivative, e.g. of Formula II, or Formula IIa, as described herein.

In some embodiments, described herein is a method comprising administering a therapeutically effective amount compound of Formula I, or a pharmaceutically acceptable salt thereof, wherein Formula I is:

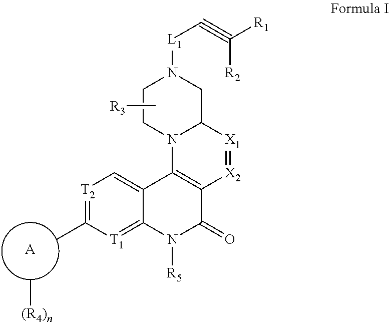

wherein,
R₁, R₂ are independently selected from H, halogen and C₁₋₆ alkyl, the C₁₋₆ alkyl is optionally substituted by 1, 2 or 3 R;
R₃ is selected from H, halogen, OH, NH₂, CN, C₁₋₆ alkyl, C₁₋₆ heteroalkyl, 3-6 membered heterocycloalkyl, C₃₋₆ cycloalkyl, 3-6 membered heterocycloalkyl-O— and C₃₋₆ cycloalkyl-O—, the C₁₋₆ alkyl, C₁₋₆ heteroalkyl, 3-6 membered heterocycloalkyl, C₃₋₆ cycloalkyl, 3-6 membered heterocycloalkyl-O— or C₃₋₆ cycloalkyl-O— is optionally substituted by 1, 2 or 3 R;
R₄ is independently selected from H, halogen, OH, NH₂, CN, C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl and 5-6 membered heteroaryl-fused 5-6 membered heterocycloalkyl, the C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
R₅ is selected from H, C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl and 5-6 membered heteroaryl-fused 5-6 membered heterocycloalkyl, the C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
L₁ is selected from —C(═O)—, —S(═O)— and —S(═O)₂—;
R₆ is selected from H, CN, C₁₋₆ alkyl, C₁₋₆ alkyl-S(═O)₂—, 3-6 membered heterocycloalkyl, —C₁₋₆ alkyl-3-6 membered heterocycloalkyl and C₃₋₆ cycloalkyl-C(═O)—, the C₁₋₆ alkyl, C₁₋₆ alkyl-S(═O)₂—, 3-6 membered heterocycloalkyl, —C₁₋₆ alkyl-3-6 membered heterocycloalkyl or C₃₋₆ cycloalkyl-C(═O)— is optionally substituted by 1, 2 or 3 R;
R₇ is independently selected from H, halogen, OH, NH₂, CN, —C(═O)OH, C₁₋₆ alkyl-O—C(═O)—, —C(═O)—NH₂, C₁₋₆ alkyl, C₁₋₆ heteroalkyl and —C₁₋₆ alkyl-3-6 membered heterocycloalkyl, the C₁₋₆ alkyl, C₁₋₆ heteroalkyl, C₁₋₆ alkyl-O—C(═O)— or —C₁₋₆ alkyl-3-6 membered heterocycloalkyl is optionally substituted by 1, 2 or 3 R;
T₁, T₂ are independently selected from N and —C(R₈)—;
R₈ is selected from H, halogen, OH, NH₂, CN, C₁₋₆ alkyl, C₁₋₆ heteroalkyl, C₃₋₆ cycloalkyl and 3-6 membered heterocycloalkyl, the C₁₋₆ alkyl, C₁₋₆ heteroalkyl, C₃₋₆ cycloalkyl or 3-6 membered heterocycloalkyl is optionally substituted by 1, 2 or 3 R;
R is independently selected from H, halogen, OH, NH₂, CN,

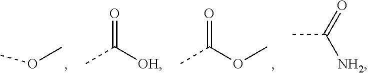

C₁₋₆ alkyl, C₁₋₆ heterocycloalkyl, C₃₋₆ cycloalkyl, 5-6 membered heterocycloalkyl, C₃₋₆ cycloalkyl-O— and 5-6 membered heterocycloalkyl-O—, the C₁₋₆ alkyl, C₁₋₆ heterocycloalkyl, C₃₋₆ cycloalkyl, 5-6 membered heterocycloalkyl, C₃₋₆ cycloalkyl-O— or 5-6 membered heterocycloalkyl-O— is optionally substituted by 1, 2 or 3 R′;
R′ is selected from F, Cl, Br, I, OH, NH₂ and CH₃;
ring A is independently selected from C₆₋₁₀ aryl, 5-10 membered heteroaryl, benzo 5-6 membered heterocycloalkyl and 5-6 membered heteroaryl-fused 5-6 membered heterocycloalkyl;
n is selected from 0, 1, 2, 3 or 4;
[symbol] is [symbol] or [symbol] , and when [symbol] is [symbol] , R₂ is not existed;
[symbol] is [symbol] or;
when in [symbol] , [symbol] is [symbol] , X₁, X₂ are independently selected from —N═, —C(R₇)═ and —C(R₇)₂—C(R₇)═;
when in [symbol] , [symbol] is [symbol] , X₁, X₂ are independently selected from single bond, —O—, —S—, S(═O), S(═O)₂, —N(R₆)—, —C(═O)—, —C(R₇)₂— and —C(R₇)₂—C(R₇)₂—;
the above 3-6 membered heterocycloalkyl, 5-6 membered heteroaryl, 5-6 membered heterocycloalkyl, 5-10 membered heteroaryl or C₁₋₆ heterocycloalkyl comprises 1, 2, or 3 heteroatoms or heteroatomic groups independently selected from —O—, —NH—, —S—, —C(═O)—, —C(═O)O—, —S(═O)—, —S(═O)₂— and N.

The compound of Formula I, or Ia, or a pharmaceutically acceptable salt thereof is a KRAS G12C inhibitor (“KRAS inhibitor”). The compound of Formula I and Formula Ia, including their synthesis, isolation, and KRAS inhibitor utility are substantially described by International Patent Application No. PCT/CN2020/116510, filed Sep. 21, 2020, which is incorporated herein by reference in its entirety.

In some embodiments, described herein is a method comprising administering a therapeutically effective amount of a SHP2 inhibitor having the structure of Formula II, or a pharmaceutically acceptable salt or solvate thereof:

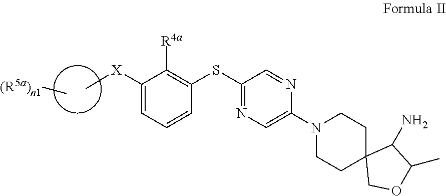

wherein.
X is selected from chemical bond, —NH—, —CONH—;
R^{4a} is selected from H, D, halogen atom, —CN, —C(O)OH, —CHO, —OH, —NO₂, —C(O)NHR^{14a} or —NHC(O)R^{15a}, substituted or unsubstituted with the group selected from —NH₂, C₁-C₁₀ alkyl, C₁-C₁₀ alkylamino, C₁-C₁₀ alkoxy, C₃-C₁₂ cycloalkyl, 3-12 membered heterocyclic group, C₆-C₁₀ aryl, or 5-10 membered heteroaryl; wherein R^{14a} and R^{15a} are each independently selected from C₁-C₁₀ alkylamino, C₃-C₁₂ cycloalkyl, C₆-C₁₀ aryl or 5-10 membered heteroaryl group; the substituent is selected from C₁-C₁₀ alkyl, halogen, —NH₂, —CN, —C(O)OH, —CHO, —OH, —NO₂, or C₁-C₁₀ alkoxy, substituted by one or more substituents of C₁-C₁₀ alkylamino, C₃-C₁₂ cycloalkyl, C₆-C₁₀ aryl, 5-10 membered heteroaryl, or 3-12 membered heterocyclic group, the substituents are optionally selected from C₁-C₁₀ alkyl, halogen, —NH₂, —CN, —C(O)OH, —CHO, —OH, —NO₂, C₁-C₁₀ alkoxy, C₁-C₁₀ alkylamino, or C₃-C₁₂ cycloalkyl;

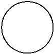

is selected from C₆-C₁₀ aryl, 5-10 membered heteroaryl, C₄-C₁₂ cycloalkyl, 3-12 membered heterocyclic group, C₆-C₁₄ bridged ring group or spiro ring group, or C₆-C₁₄ bridged heterocyclic group or spiro heterocyclic group; wherein the 5-10 membered heteroaryl group, 3-12 membered heterocyclic group, C₆-C₁₄ bridged heterocyclic group or spiro heterocyclic group contains one to three heteroatom or groups selected from N, NH, O, S, C(O), or S(O);
each R^{5a} is the same or different, and is independently selected from H, D, halogen atom, —CN, —C(O)OH, —CHO, —OH, —NO₂, or aminoacyl, substituted or unsubstituted with the group selected from C₁-C₁₀ alkyl, C₁-C₁₀ alkylamino, C₁-C₁₀ alkoxy, —NH₂, C₃-C₁₂ cycloalkyl, 3-12 membered heterocyclic group, C₆-C₁₀ aryl or 5-10 membered heteroaryl, the substituent selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaromatic group, C₆-C₁₀ aryl or 3-12 membered heterocyclic group substituted by one or more substituents; or any two adjacent R^{5a} form a 3-6 membered saturated or unsaturated ring, and is optionally, the 3-6 membered saturated or unsaturated ring is comprises one to three —OH, —NH₂, —CN, halogen, C₁-C₁₀ alkyl, C₁-C₁₀ alkoxy, C₃-C₁₂ cycloalkylamino, C₁-C₁₀ alkylamino, C₃-C₁₂ cycloalkyl, halogenated C₁-C₁₀ alkylamino, C₆-C₁₀ aryl or 5-10 member heteroaryl; and
n1 is 0, 1, 2 or 3.

In some embodiments, R^{4a} is selected from H, D, halogen, —CN, unsubstituted or halogen atom substituted C₁-C₁₀ alkyl.

In some embodiments,

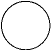

is elected from phenyl, naphthyl, 5-10 membered heteroaryl or 3-12 membered heterocyclic group; wherein the 5-10 membered heteroaryl group and 3-12 membered heterocyclic group contain one to three heteroatoms or groups optionally selected from N, NH, O, S, or C(O).

In some embodiments, the 5-10 membered heteroaromatic ring is selected from thienyl; pyridyl; pyrimidinyl; pyrazinyl; pyridazinyl; pyrrolyl; pyrazolyl; thiazolyl; 1,2,3-triazolyl; 1,2,4-triazolyl; imidazolyl; tetrazolyl; isothiazolyl; oxazolyl; isoxazolyl; thiadiazolyl; oxadiazolyl; benzothienyl; indolyl; benzimidazolyl; benzothiazolyl; benzofuranyl; quinolinyl; isoquinolinyl; quinazolinyl; indazolyl; indole[1,2-a]pyrazinyl; 4,7-diazaindole; pyrazolopyrimidinyl; imidazo-pyrimidinyl; oxazolopyrimidinyl; isoxazopyrimidiny; imidazopyrazinyl; pyrazolopyrazine; pyrrolopyrazinyl; or furan. In some embodiments, any one of pyrazinyl, thienopyrazinyl, pyridopyrimidinone, benzoxazolyl, and benzothiazolyl; the 3-12 membered heterocyclic group is selected from aziridinyl, oxetanyl, pyrrolidinyl, tetrahydrofuranyl, tetrahydrothienyl, piperidinyl, morpholinyl, piperazinyl, thiomorpholinyl, tetrahydropyranyl, 1,1-dioxythiomorpholinyl, butyrolactam, valerolactam, caprolactam, butyrolactone, valerolactone, caprolactone, succinimide or

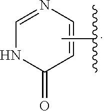

In some embodiments, the 3-12 membered heterocyclic group is selected from butyrolactamyl, pyrrolidinyl, succinimide, or

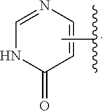

In some embodiments, each R^{5a} is the same or different, and is independently selected from H, D, halogen, —CN, —C(O)OH, —CHO, —OH, —NO₂, or aminoacyl, substituted or unsubstituted with a C₁-C₁₀ alkyl, C₁-C₁₀ alkylamino, C₁-C₁₀ alkoxy, or —NH₂, and the substitution is selected from C₁-C₁₀ alkyl, halogen, —NH₂, —CN, —OH, —NO₂ are substituted by one or more substituents; or any two adjacent R^{5a} form a 3-6 membered saturated or unsaturated ring, optionally, the 3-6-membered saturated or unsaturated ring is substituted with one to three —OH, —NH₂, —CN, halogen, C₁-C₁₀ alkyl, and C₁-C₁₀ alkoxy.

In some embodiments, the compound of Formula II has the structure of Formula IIa, or a pharmaceutically acceptable salt or solvate thereof, wherein Formula IIa is:

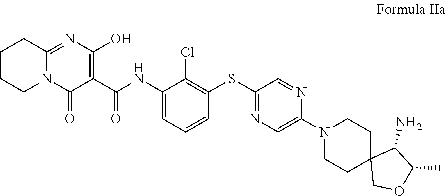 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

In some embodiments, the compound of Formula II or IIa is N-(3-((5-((3S,4S)-4-amino-3-methyl-2-oxa-8-azaspiro[4.5]decan-8-yl)pyrazin-2-yl)thio)-2-chlorophenyl)-2-hydroxy-4-oxo-6,7,8,9-tetrahydro-4H-pyrido[1,2-a]pyrimidine-3-carboxamide, or a pharmaceutically acceptable salt or solvate thereof. The compound of Formula IIa is also referred to herein as HBI-2376.

The compound of Formula II, or IIa, or a pharmaceutically acceptable salt thereof is a SHP2 inhibitor. The compound of Formula II and Formula IIa, including their synthesis, isolation, and SHP2 inhibitor activity, are substantially described by International Patent Application No. PCT/CN2020/077391, filed Mar. 2, 2020, which is incorporated herein by reference in its entirety.

Some embodiments described herein provide a method of treating cancer, comprising administering to the subject having a tumor an effective amount of a combination comprising an amount of a Kirsten Rat Sarcoma oncogene homologue G12C (KRAS) inhibitor and an amount of a SH2-containing protein tyrosine phosphatase 2 (SHP2) inhibitor, wherein the KRAS inhibitor is a compound of Formula I, or a pharmaceutically acceptable salt thereof, and the SHP2 inhibitor is a compound of Formula II, or a pharmaceutically acceptable salt thereof,

In some embodiments of the method recited in the immediately preceding paragraph, Formula I is:

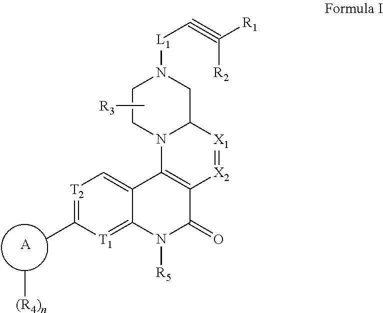

wherein:
   R₁, R₂ are independently selected from H, halogen and C₁₋₆ alkyl, the C₁₋₆ alkyl is optionally substituted by 1, 2 or 3 R;
   R₃ is selected from H, halogen, OH, NH₂, CN, C₁₋₆ alkyl, C₁₋₆ heteroalkyl, 3-6 membered heterocycloalkyl, C₃₋₆ cycloalkyl, 3-6 membered heterocycloalkyl-O— and C₃₋₆ cycloalkyl-O—, the C₁₋₆ alkyl, C₁₋₆ heteroalkyl, 3-6 membered heterocycloalkyl, C₃₋₆ cycloalkyl, 3-6 membered heterocycloalkyl-O— or C₃₋₆ cycloalkyl-O— is optionally substituted by 1, 2 or 3 R;
   R₄ is independently selected from H, halogen, OH, NH₂, CN, C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl and 5-6 membered heteroaryl-fused 5-6 membered heterocycloalkyl, the C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
   R₅ is selected from H, C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl and 5-6 membered heteroaryl-fused 5-6 membered heterocycloalkyl, the C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
   L₁ is selected from —C(═O)—, —S(═O)— and —S(═O)₂—;
   R₆ is selected from H, CN, C₁₋₆ alkyl, C₁₋₆ alkyl-S(═O)₂—, 3-6 membered heterocycloalkyl, —C₁₋₆ alkyl-3-6 membered heterocycloalkyl and C₃₋₆ cycloalkyl-C(═O)—, the C₁₋₆ alkyl, C₁₋₆ alkyl-S(═O)₂—, 3-6 membered heterocycloalkyl, —C₁₋₆ alkyl-3-6 membered heterocycloalkyl or C₃₋₆ cycloalkyl-C(═O)— is optionally substituted by 1, 2 or 3 R;
   R₇ is independently selected from H, halogen, OH, NH₂, CN, —C(═O)OH, C₁₋₆ alkyl-O—C(═O)—, —C(═O)—NH₂, C₁₋₆ alkyl, C₁₋₆ heteroalkyl and —C₁₋₆ alkyl-3-6 membered heterocycloalkyl, the C₁₋₆ alkyl, C₁₋₆ heteroalkyl, C₁₋₆ alkyl-O—C(═O)— or —C₁₋₆ alkyl-3-6 membered heterocycloalkyl is optionally substituted by 1, 2 or 3 R;
   T₁, T₂ are independently selected from N and —C(R₈)—;
   R₈ is selected from H, halogen, OH, NH₂, CN, C₁₋₆ alkyl, C₁₋₆ heteroalkyl, C₃₋₆ cycloalkyl and 3-6 membered heterocycloalkyl, the C₁₋₆ alkyl, C₁₋₆ heteroalkyl, C₃₋₆ cycloalkyl or 3-6 membered heterocycloalkyl is optionally substituted by 1, 2 or 3 R;
   R is independently selected from H, halogen, OH, NH₂, CN,

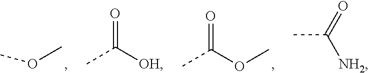

C₁₋₆ alkyl, C₁₋₆ heterocycloalkyl, C₃₋₆ cycloalkyl, 5-6 membered heterocycloalkyl, C₃₋₆ cycloalkyl-O— and 5-6 membered heterocycloalkyl-O—, the C₁₋₆ alkyl, C₁₋₆ heterocycloalkyl, C₃₋₆ cycloalkyl, 5-6 membered heterocycloalkyl, C₃₋₆ cycloalkyl-O— or 5-6 membered heterocycloalkyl-O— is optionally substituted by 1, 2 or 3 R′;
R′ is selected from F, Cl, Br, I, OH, NH₂ and CH₃;
ring A is independently selected from C₆₋₁₀ aryl, 5-10 membered heteroaryl, benzo 5-6 membered heterocycloalkyl and 5-6 membered heteroaryl-fused 5-6 membered heterocycloalkyl;
n is selected from 0, 1, 2, 3 or 4;
[symbol] is [symbol] or [symbol] , and when [symbol] is [symbol] , R₂ is not existed;
[symbol] is [symbol] or;
when in [symbol] , [symbol] is [symbol] , X₁, X₂ are independently selected from —N═, —C(R₇)═ and —C(R₇)₂—C(R₇)═;
when in [symbol] , [symbol] is [symbol] , X₁, X₂ are independently selected from single bond, —O—, —S—, S(═O), S(═O)₂, —N(R₆)—, —C(═O)—, —C(R₇)₂— and —C(R₇)₂—C(R₇)₂—;
   the above 3-6 membered heterocycloalkyl, 5-6 membered heteroaryl, 5-6 membered heterocycloalkyl, 5-10 membered heteroaryl or C₁₋₆ heterocycloalkyl comprises 1, 2, or 3 heteroatoms or heteroatomic groups independently selected from —O—, —NH—, —S—, —C(═O)—, —C(═O)O—, —S(═O)—, —S(═O)₂— and N;
   and
   Formula II is:

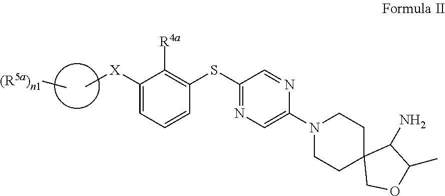

wherein:
   X is selected from chemical bond, —NH—, —CONH—;
   R^{4a} is selected from H, D, halogen atom, —CN, —C(O)OH, —CHO, —OH, —NO₂, —C(O)NHR^{14a} or —NHC(O)R^{15a}, substituted or unsubstituted with the group selected from —NH₂, C₁-C₁₀ alkyl, C₁-C₁₀ alkylamino, C₁-C₁₀ alkoxy, C₃-C₁₂ cycloalkyl, 3-12 membered heterocyclic group, C₆-C₁₀ aryl, or 5-10 membered heteroaryl; wherein R^{14a} and R^{15a} are each independently selected from C₁-C₁₀ alkylamino, C₃-C₁₂ cycloalkyl, C₆-C₁₀ aryl or 5-10 membered heteroaryl group; the substituent is selected from C₁-C₁₀ alkyl, halogen, —NH₂, —CN, —C(O)OH, —CHO, —OH, —NO₂, or C₁-C₁₀ alkoxy, substituted by one or more substituents of C₁-C₁₀ alkylamino, C₃-C₁₂ cycloalkyl, C₆-C₁₀ aryl, 5-10 membered heteroaryl, or 3-12 membered heterocyclic group, the substituents are optionally selected from C₁-C₁₀ alkyl, halogen, —NH₂, —CN, —C(O)OH, —CHO, —OH, —NO₂, C₁-C₁₀ alkoxy, C₁-C₁₀ alkylamino, or C₃-C₁₂ cycloalkyl;

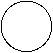

is selected from C₆-C₁₀ aryl, 5-10 membered heteroaryl, C₄-C₁₂ cycloalkyl, 3-12 membered heterocyclic group, C₆-C₁₄ bridged ring group or spiro ring group, or C₆-C₁₄ bridged heterocyclic group or spiro heterocyclic group; wherein the 5-10 membered heteroaryl group, 3-12 membered heterocyclic group, C₆-C₁₄ bridged heterocyclic group or spiro heterocyclic group contains one to three heteroatom or groups selected from N, NH, O, S, C(O), or S(O);
each R^{5a} is the same or different, and is independently selected from H, D, halogen atom, —CN, —C(O)OH, —CHO, —OH, —NO₂, or aminoacyl, substituted or unsubstituted with the group selected from C₁-C₁₀ alkyl, C₁-C₁₀ alkylamino, C₁-C₁₀ alkoxy, —NH₂, C₃-C₁₂ cycloalkyl, 3-12 membered heterocyclic group, C₆-C₁₀ aryl or 5-10 membered heteroaryl, the substituent selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaromatic group, C₆-C₁₀ aryl or 3-12 membered heterocyclic group substituted by one or more substituents; or any two adjacent R^{5a} form a 3-6 membered saturated or unsaturated ring, and is optionally, the 3-6 membered saturated or unsaturated ring is comprises one to three —OH, —NH₂, —CN, halogen, C₁-C₁₀ alkyl, C₁-C₁₀ alkoxy, C₃-C₁₂ cycloalkylamino, C₁-C₁₀ alkylamino, C₃-C₁₂ cycloalkyl, halogenated C₁-C₁₀ alkylamino, C₆-C₁₀ aryl or 5-10 member heteroaryl; and n1 is 0, 1, 2 or 3.

In some embodiments of either of the immediately preceding two paragraphs, the method comprises treating metastasis of a tumor. In some such embodiments, treating the tumor comprises treatment of metastasis of the tumor to a brain. In some such embodiments, treatment of metastasis of tumor to the brain comprises suppression of growth of a brain tumor and the effective amount of the compound is an amount effective to suppress growth of the brain tumor. In some such embodiments, the brain tumor is a secondary tumor. In some such embodiments, the primary tumor is not a brain tumor. In some such embodiments, the compound of Formula I penetrates the blood-brain barrier (BBB). In some such embodiments, the compound of Formula I is the compound of Formula Ia:

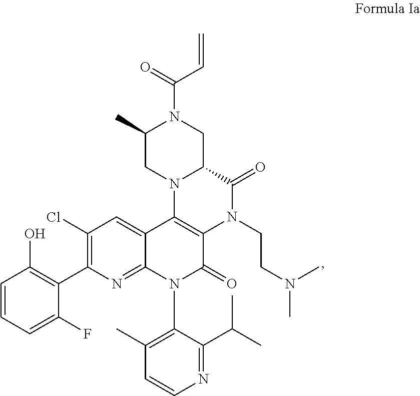 [SMILES: C=CC(=O)N1C[C@@H]2C(=O)N(CCN(C)C)c3c(c4cc(Cl)c(-c5c(O)cccc5F)nc4n(-c4c(C)ccnc4C(C)C)c3=O)N2C[C@H]1C]

or an enantiomer or a pharmaceutically acceptable salt thereof.

In some embodiments of any of the immediately preceding three paragraphs, the compound of Formula I or Formula Ia is administered to the subject in an amount of about 1 to about 100 mg/kg per day for at least one day. In some such embodiments, the amount of the compound of Formula II is synergistic with the amount of the compound of Formula I. In some such embodiments, the combined amounts of the compound of Formula I and Formula II are effective to suppress growth or induce regression of a brain tumor. In some such embodiments, the combined amounts of the compounds of Formula I and Formula II are effective to induce regression of a brain tumor. In some such embodiments, the brain tumor is a secondary tumor. In some such embodiments, the primary tumor is not a brain tumor. In some such embodiments, the compound of Formula I penetrates the blood-brain barrier (BBB).

In some embodiments of any of the immediately preceding four paragraphs, the compound of Formula II is the compound of Formula IIa, or a pharmaceutically acceptable salt or racemate thereof, wherein Formula IIa is:

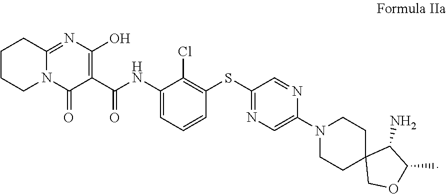 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

In some embodiments of any of the immediately preceding five paragraphs, the compound Formula IIa or a pharmaceutically acceptable salt thereof, is administered to said patient in need from about 5 mg/kg to about 25 mg/kg. In some such embodiments, the compound of Formula I is administered to the subject in an amount of about 0.5 to about 100 mg/kg per day for at least one day.

Some embodiments described herein provide a method of treating or preventing brain metastasis, comprising administering to the subject having a primary tumor an effective amount of a compound of Formula I, or a pharmaceutically acceptable salt thereof, wherein the compound of Formula I is selected from:

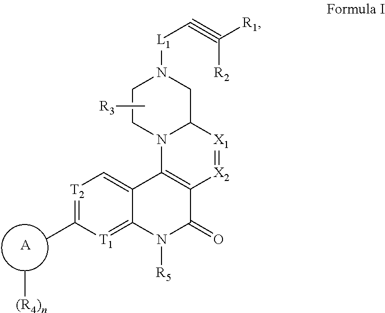

wherein:
R₁, R₂ are independently selected from H, halogen and C₁₋₆ alkyl, the C₁₋₆ alkyl is optionally substituted by 1, 2 or 3 R;
R₃ is selected from H, halogen, OH, NH₂, CN, C₁₋₆ alkyl, C₁₋₆ heteroalkyl, 3-6 membered heterocycloalkyl, C₃₋₆ cycloalkyl, 3-6 membered heterocycloalkyl-O— and C₃₋₆ cycloalkyl-O—, the C₁₋₆ alkyl, C₁₋₆ heteroalkyl, 3-6 membered heterocycloalkyl, C₃₋₆ cycloalkyl, 3-6 membered heterocycloalkyl-O— or C₃₋₆ cycloalkyl-O— is optionally substituted by 1, 2 or 3 R;
R₄ is independently selected from H, halogen, OH, NH₂, CN, C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl and 5-6 membered heteroaryl-fused 5-6 membered heterocycloalkyl, the C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
R₅ is selected from H, C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl and 5-6 membered heteroaryl-fused 5-6 membered heterocycloalkyl, the C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
L₁ is selected from —C(═O)—, —S(═O)— and —S(═O)₂—;
R₆ is selected from H, CN, C₁₋₆ alkyl, C₁₋₆ alkyl-S(═O)₂—, 3-6 membered heterocycloalkyl, —C₁₋₆ alkyl-3-6 membered heterocycloalkyl and C₃₋₆ cycloalkyl-C(═O)—, the C₁₋₆ alkyl, C₁₋₆ alkyl-S(═O)₂—, 3-6 membered heterocycloalkyl, —C₁₋₆ alkyl-3-6 membered heterocycloalkyl or C₃₋₆ cycloalkyl-C(═O)— is optionally substituted by 1, 2 or 3 R;
R₇ is independently selected from H, halogen, OH, NH₂, CN, —C(═O)OH, C₁₋₆ alkyl-O—C(═O)—, —C(═O)—NH₂, C₁₋₆ alkyl, C₁₋₆ heteroalkyl and —C₁₋₆ alkyl-3-6 membered heterocycloalkyl, the C₁₋₆ alkyl, C₁₋₆ heteroalkyl, C₁₋₆ alkyl-O—C(═O)— or —C₁₋₆ alkyl-3-6 membered heterocycloalkyl is optionally substituted by 1, 2 or 3 R;
T₁, T₂ are independently selected from N and —C(R₈)—;
R₈ is selected from H, halogen, OH, NH₂, CN, C₁₋₆ alkyl, C₁₋₆ heteroalkyl, C₃₋₆ cycloalkyl and 3-6 membered heterocycloalkyl, the C₁₋₆ alkyl, C₁₋₆ heteroalkyl, C₃₋₆ cycloalkyl or 3-6 membered heterocycloalkyl is optionally substituted by 1, 2 or 3 R;
R is independently selected from H, halogen, OH, NH₂, CN,

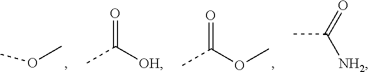

C₁₋₆ alkyl, C₁₋₆ heterocycloalkyl, C₃₋₆ cycloalkyl, 5-6 membered heterocycloalkyl, C₃₋₆ cycloalkyl-O— and 5-6 membered heterocycloalkyl-O—, the C₁₋₆ alkyl, C₁₋₆ heterocycloalkyl, C₃₋₆ cycloalkyl, 5-6 membered heterocycloalkyl, C₃₋₆ cycloalkyl-O— or 5-6 membered heterocycloalkyl-O— is optionally substituted by 1, 2 or 3 R′;
R′ is selected from F, Cl, Br, I, OH, NH₂ and CH₃;
ring A is independently selected from C₆₋₁₀ aryl, 5-10 membered heteroaryl, benzo 5-6 membered heterocycloalkyl and 5-6 membered heteroaryl-fused 5-6 membered heterocycloalkyl;
n is selected from 0, 1, 2, 3 or 4;
[symbol] is [symbol] or [symbol] , and when [symbol] is [symbol] , R₂ is not existed;
[symbol] is [symbol] or;
when in [symbol] , [symbol] is [symbol] , X₁, X₂ are independently selected from —N═, —C(R₇)═ and —C(R₇)₂—C(R₇)═;
when in [symbol] , [symbol] is [symbol] , X₁, X₂ are independently selected from single bond, —O—, —S—, S(═O), S(═O)₂, —N(R₆)—, —C(═O)—, —C(R₇)₂— and —C(R₇)₂—C(R₇)₂—;
the above 3-6 membered heterocycloalkyl, 5-6 membered heteroaryl, 5-6 membered heterocycloalkyl, 5-10 membered heteroaryl or C₁₋₆ heterocycloalkyl comprises 1, 2, or 3 heteroatoms or heteroatomic groups independently selected from —O—, —NH—, —S—, —C(═O)—, —C(═O)O—, —S(═O)—, —S(═O)₂— and N.

In some embodiments of the immediately preceding paragraph, treating brain metastasis comprises suppression of growth of a brain tumor and the effective amount of the compound is an amount effective to suppress growth of a brain tumor. In some such embodiments, the brain tumor is a secondary tumor. In some such embodiments, the primary tumor is not a brain tumor. In some such embodiments, the compound of Formula I penetrates the blood-brain barrier (BBB). In some such embodiments, the compound of Formula I is the compound of formula Ia:

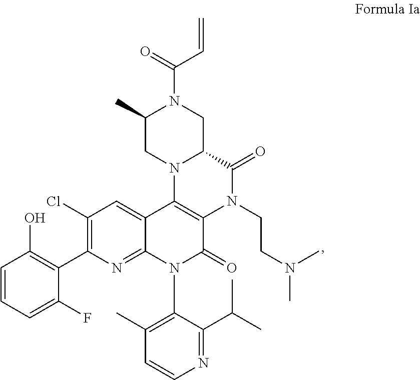 [SMILES: C=CC(=O)N1C[C@@H]2C(=O)N(CCN(C)C)c3c(c4cc(Cl)c(-c5c(O)cccc5F)nc4n(-c4c(C)ccnc4C(C)C)c3=O)N2C[C@H]1C]

or an enantiomer or a pharmaceutically acceptable salt thereof.

In some embodiments of the immediately preceding two paragraphs, the compound of Formula I or Formula Ia is administered to the subject in an amount of about 1 to about 100 mg/kg per day for at least one day. Some such embodiments comprise administering an effective amount a compound of Formula II, or a pharmaceutically acceptable salt or enantiomer thereof, wherein the compound of Formula II is:

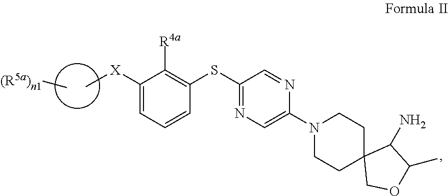

wherein:
X is selected from chemical bond, —NH—, —CONH—;
R^{4a} is selected from H, D, halogen atom, —CN, —C(O)OH, —CHO, —OH, —NO₂, —C(O)NHR^{14a} or —NHC(O)R^{15a}, substituted or unsubstituted with the group selected from —NH₂, C₁-C₁₀ alkyl, C₁-C₁₀ alkylamino, C₁-C₁₀ alkoxy, C₃-C₁₂ cycloalkyl, 3-12 membered heterocyclic group, C₆-C₁₀ aryl, or 5-10 membered heteroaryl; wherein R^{14a} and R^{15a} are each independently selected from C₁-C₁₀ alkylamino, C₃-C₁₂ cycloalkyl, C₆-C₁₀ aryl or 5-10 membered heteroaryl group; the substituent is selected from C₁-C₁₀ alkyl, halogen, —NH₂, —CN, —C(O)OH, —CHO, —OH, —NO₂, or C₁-C₁₀ alkoxy, substituted by one or more substituents of C₁-C₁₀ alkylamino, C₃-C₁₂ cycloalkyl, C₆-C₁₀ aryl, 5-10 membered heteroaryl, or 3-12 membered heterocyclic group, the substituents are optionally selected from C₁-C₁₀ alkyl, halogen, —NH₂, —CN, —C(O)OH, —CHO, —OH, —NO₂, C₁-C₁₀ alkoxy, C₁-C₁₀ alkylamino, or C₃-C₁₂ cycloalkyl;

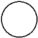

is selected from C₆-C₁₀ aryl, 5-10 membered heteroaryl, C₄-C₁₂ cycloalkyl, 3-12 membered heterocyclic group, C₆-C₁₄ bridged ring group or spiro ring group, or C₆-C₁₄ bridged heterocyclic group or spiro heterocyclic group; wherein the 5-10 membered heteroaryl group, 3-12 membered heterocyclic group, C₆-C₁₄ bridged heterocyclic group or spiro heterocyclic group contains one to three heteroatom or groups selected from N, NH, O, S, C(O), or S(O);
each R^{5a} is the same or different, and is independently selected from H, D, halogen atom, —CN, —C(O)OH, —CHO, —OH, —NO₂, or aminoacyl, substituted or unsubstituted with the group selected from C₁-C₁₀ alkyl, C₁-C₁₀ alkylamino, C₁-C₁₀ alkoxy, —NH₂, C₃-C₁₂ cycloalkyl, 3-12 membered heterocyclic group, C₆-C₁₀ aryl or 5-10 membered heteroaryl, the substituent selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaromatic group, C₆-C₁₀ aryl or 3-12 membered heterocyclic group substituted by one or more substituents; or any two adjacent R^{5a} form a 3-6 membered saturated or unsaturated ring, and is optionally, the 3-6 membered saturated or unsaturated ring is comprises one to three —OH, —NH₂, —CN, halogen, C₁-C₁₀ alkyl, C₁-C₁₀ alkoxy, C₃-C₁₂ cycloalkylamino, C₁-C₁₀ alkylamino, C₃-C₁₂ cycloalkyl, halogenated C₁-C₁₀ alkylamino, C₆-C₁₀ aryl or 5-10 member heteroaryl; and
n1 is 0, 1, 2 or 3.

In some embodiments of the immediately preceding three paragraphs, the amount of the compound of Formula II is synergistic with the amount of the compound of Formula I. In some such embodiments, the combined amounts of the compound of Formula I and Formula II are effective to suppress growth or induce regression of a brain tumor. In some such embodiments, the combined amounts of the compounds of Formula I and Formula II are effective to induce regression of a brain tumor. In some such embodiments, the brain tumor is a secondary tumor. In some such embodiments, the primary tumor is not a brain tumor. In some such embodiments, the compound of Formula I penetrates the blood-brain barrier (BBB). In some such embodiments, the compound of Formula I is a compound of Formula Ia:

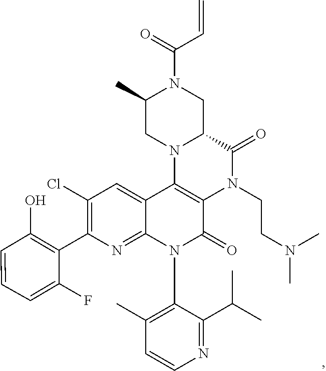 [SMILES: C=CC(=O)N1C[C@@H]2C(=O)N(CCN(C)C)c3c(c4cc(Cl)c(-c5c(O)cccc5F)nc4n(-c4c(C)ccnc4C(C)C)c3=O)N2C[C@H]1C]

or an enantiomer or a pharmaceutically acceptable salt thereof.

In some embodiments of any of the immediately preceding four paragraphs, the compound of Formula I is administered to the subject in an amount of about 1 to about 100 mg/kg per day for at least one day. In some such embodiments, the compound of Formula II is the compound of Formula IIa, or a racemate or pharmaceutically acceptable salt thereof, wherein Formula II is:

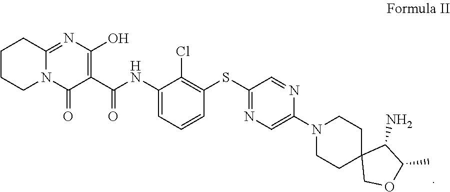 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

In some embodiments of any of the immediately preceding five paragraphs, the compound Formula IIa or a pharmaceutically acceptable salt thereof, is administered to said patient in need from about 5 mg/kg to about 25 mg/kg. In some such embodiments, the compound of Formula I is administered to the subject in an amount of about 0.5 to about 100 mg/kg per day for at least one day. In some such embodiments, the method is used to treat cancer in the said patient.

The invention thus comprises combinations of a KRAS inhibitor and a SNP2 inhibitor. The invention also comprises combinations of a KRAS inhibitor and a SNP2 inhibitor for treatment of cancer, in particular metastatic cancer, and especially metastatic cancer that has metastasized to the brain. In some such embodiments, the KRAS inhibitor is a compound, or pharmaceutically acceptable salt thereof, of Formula I or Formula Ia, as described herein and, in particular, in any of the immediately preceding six paragraphs. In some such embodiments, the SNP2 inhibitor is a compound, or pharmaceutically acceptable salt thereof, of Formula II or Formula IIa, as described herein, and in particular in any of the preceding six paragraphs. Thus, the invention described herein also includes the use of any combination described herein, and in particular in any of the immediately preceding six paragraphs for the treatment of cancer, specifically metastatic cancer, and more particularly metastatic cancer that has metastasized to the brain. Thus, the invention described herein teaches combinations, uses, and methods for treatment of secondary brain tumor, especially such secondary brain tumor whose original tumor lay outside the brain.

In some embodiments, the method comprises compounds as described herein, or a pharmaceutically acceptable salts or solvates thereof (e.g., Formula (Ia or IIa)) present at an amount of greater than about 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. In some other embodiments, the compounds described herein are present in an amount greater than about 5 mg or about 10 mg. In some embodiments, the composition comprises compounds described herein in an amount from about 1 mg to about 500 mg. In some embodiments, the composition comprises compounds described herein in an amount from about 1 mg to about 10 mg, from about 1 mg to about 25 mg, from about 1 mg to about 50 mg, from about 5 mg to about 10 mg, from about 5 mg to about 25 mg, from about 5 mg to about 50 mg, from about 10 mg to about 25 mg, from about 10 mg to about 50 mg, from about 50 mg to about 100 mg, from about 100 mg to about 200 mg, or from about 200 mg to about 500 mg.

In some embodiments, the combination comprises at least about 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg individually of the compounds described herein, or a pharmaceutically acceptable salt or solvate thereof. In some embodiments, the combination comprises at least about 1 mg, 2 mg, 3 mg, 4 mg, 5 mg, 6 mg, 7 mg, 8 mg, 9 mg, or 10 mg individually of the compounds described herein. In some embodiments, the compounds described herein are present in the composition in an amount of at least about 5 mg or about 10 mg. In some embodiments, the combination comprises at least about 1 mg to about 10 mg, about 1 mg to about 25 mg, about 1 mg to about 50 mg, about 5 mg to about 10 mg, about 5 mg to about 25 mg, about 5 mg to about 50 mg, about 10 mg to about 25 mg, about 10 mg to about 50 mg, about 50 mg to about 100 mg, or about 100 mg to about 200 mg of the compounds described herein.

In some embodiments, the combination comprises from about 5 mg to about 500 mg or from about 5 mg to about 100 mg of the compounds described herein, or a pharmaceutically acceptable salt or solvate thereof. In some embodiments, the combination comprises about 5 mg, about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, or about 200 mg of the compounds described herein.

In some embodiments, the method comprises compounds described herein, or a pharmaceutically acceptable salt or solvate thereof (e.g., Formula (Ia or IIa)) in an amount relative to the weight of the patient (i.e., mg/kg). In some instances, the compounds described herein are present in an amount equivalent to about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg. In other instances the compounds described herein are present in an amount equivalent to about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg.

In some embodiments, the combination comprises from about 5 mg/kg to about 25 mg/kg per patient body weight of a compound described herein, or a pharmaceutically acceptable salt or solvate thereof (e.g. a compound of Formula Ia or Formula IIa). In some embodiments, the combination comprises about 5 mg/kg, about 10 mg/kg, about 15 mg/kg, about 20 mg/kg, or about 25 mg/kg per body weight of a compound described herein.

In some embodiments, the compounds as described herein is provided in amounts that are synergistic. The term synergistic refers to a combination described herein (e.g., a compound of Formula Ia and compound of formula IIa, including coadministration with another active agent such as an anti-cancer agent described herein) or a combination of regimens that is more effective than the additive effects of each individual therapy or regimen.

A synergistic effect of a combination described herein can permit the use of lower dosages of one or more of the components of the combination (e.g., a compound of Formula Ia or a compound of Formula IIa). A synergistic effect can permit less frequent administration of at least one of the administered therapies (e.g., a compound of Formula Ia or a compound of Formula IIa) to a subject with a disease, disorder, or condition described herein. Such lower dosages and reduced frequency of administration can reduce the toxicity associated with the administration of at least one of the therapies to a subject without reducing the efficacy of the treatment. A synergistic effect avoids or reduces adverse unwanted side effects associated with the use of any therapy.

In some embodiments, the compounds described herein are metabolized upon administration, the formed metabolite is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexyl-amine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Pharmaceutical Compositions

In one aspect, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the pharmaceutical formulation is in the form of a tablet. In other embodiments, pharmaceutical formulations of the compounds disclosed herein are in the form of a capsule.

In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In some embodiments, a compound disclosed herein is formulated into a pharmaceutical composition suitable for intramuscular, subcutaneous, or intravenous injection.

In some embodiments, the compound disclosed herein is be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

Pharmaceutical compositions and dosage forms described herein typically include one or more excipients. Suitable excipients are well known to those skilled in the art of pharmacy. Whether a certain excipient is suitable for incorporation into a pharmaceutical composition or dosage form depends on a variety of factors such as, for example, the intended route of administration to the patient. Pharmaceutical compositions described herein can include other agents such as stabilizers, lubricants, buffers, and disintegrants that can reduce the rate by which an active ingredient can decompose in a certain formulation.

Pharmaceutical compositions described herein can in certain instances include additional active agents other than those in the combinations described herein (e.g., an anti-cancer agent such as those described herein) in an amount provided herein.

In some embodiments, the compounds described herein are provided in an oral dosage form such as a tablet or capsule. In some embodiment, the compounds described herein are supplied as a powder (e.g., lyophilized powder) that can be resuspended in a liquid suitable for parenteral administration.

Combinations described herein can be provided as controlled release pharmaceutical products, which have a goal of improving drug therapy over that achieved by their non-controlled counterparts. Controlled release formulations can extend activity of the drug, reduce dosage frequency, and increase subject compliance. In addition, controlled release formulations can be used to affect the time of onset of action or other characteristics, such as blood levels of the drug, and can thus affect the occurrence of side (e.g., adverse) effects.

### Methods of Treatment

The combinations and/or the pharmaceutical compositions described herein are useful for treating diseases, disorders, or alleviating or eliminating the symptoms of diseases and disorders such as, for example, cancer.

In some embodiments, described herein is a method of treating cancer in a patient in need thereof, the method comprising administering to the patient a KRAS inhibitor and a SHP2 inhibitor compound as described herein, or a pharmaceutically acceptable salt or solvate thereof (e.g. compounds of Formula Ia and Formula IIa). In some other embodiments, described herein is a method of treating brain metastasis in a patient in need thereof, the method comprising administering to the patient a KRAS inhibitor as described herein, or a pharmaceutically acceptable salt or solvate thereof (e.g. compounds of Formula Ia).

In some embodiments, described herein is a method of treating brain metastasis in a patient in need thereof, the method comprising administering to the patient a KRAS inhibitor and a SHP2 inhibitor compound as described herein, or a pharmaceutically acceptable salt or solvate thereof (e.g. compounds of Formula Ia and Formula IIa).

In some embodiments, the cancer is in the form of a tumor. In some embodiments, the cancer is selected from squamous cell carcinoma, non-squamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, brain tumor, colorectal cancer, pancreatic cancer, lymphoma, leiomyosarcoma, liposarcoma, synovial sarcoma, or malignant peripheral sheath tumor (MPNST). In some embodiments, the cancer is squamous cell carcinoma. In some embodiments, the cancer is non-squamous cell carcinoma. In some embodiments, the cancer is non-small cell lung cancer (NSCLC). In some embodiments, the cancer is small cell lung cancer. In some embodiments, the cancer is melanoma. In some embodiments, the cancer is hepatocellular carcinoma. In some embodiments, the cancer is renal cell carcinoma. In some embodiments, the cancer is ovarian cancer. In some embodiments, the caner is head and neck cancer. In some embodiments, the cancer is urothelial cancer. In some embodiments, the cancer is breast cancer (e.g., HER2 negative or HER2 positive breast cancer). In some embodiments, the cancer is prostate cancer. In some embodiments, the cancer is glioblastoma. In some embodiments, the cancer is colorectal cancer. In some embodiments, the care is pancreatic cancer. In some embodiments, the cancer is lymphoma. In some embodiments, the cancer is synovial sarcoma. In some embodiments, the cancer is malignant peripheral sheath tumor (MPNST).

In some embodiments, the tumor is a solid tumor. In some embodiments, the method of treating cancer reduces the tumor volume or tumor burden in the patient. In some embodiments, the tumor is reduced in volume from 5% to 95% or 5% to 50% or any value therein. In some embodiments, the tumor is reduced in volume by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the tumor is reduced in volume by at least 5%, at least 10%, at least 20%, at least 30%, at least 40%, or at least 50%. %. In some embodiments, the tumor is reduced by about 10% to about 99%. In some embodiments, the tumor is reduced by about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99%. In some embodiments, the tumor is reduced by about 10%, about 20%, about 30%, about 40%, about 50%, about 60%, about 70%, about 80%, about 90%, or about 99%. In some embodiments, the tumor is reduced by at least about 10%, about 20%, about 30%, about 40%, about 50%, about 60%, about 70%, about 80%, or about 90%. In some embodiments, the tumor is reduced by at most about 20%, about 30%, about 40%, about 50%, about 60%, about 70%, about 80%, about 90%, or about 99%.

In some embodiments, the cancer is a hematological cancer. In some embodiments, the cancer is a hematological cancer selected from lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CML), acute lymphocytic leukemia (ALL), or chronic lymphocytic leukemia (CLL). In some embodiments, the cancer is Hodgkin's Lymphoma or Reed-Sternberg disease.

In some embodiments, the cancer is a Stage I, Stage II, Stage, III, or Stage IV cancer. In some embodiments, the cancer is a Stage I cancer (e.g., Stage IA, IB, or IC). In some embodiments, the cancer is a Stage II cancer (e.g., Stage IIA or IIB). In some embodiments, the cancer is a Stage III cancer, (e.g., Stage IIIA, IIIB, or IIIC). In some embodiments, the cancer is a Stage IV cancer, (e.g., Stage IVA or IVB). In some other embodiments, the cancer has metastasized.

The methods described herein can be administered to a cancer patient at any time following diagnosis. For example, the cancer patient can be treatment naive (i.e., has not received a cancer therapy for the diagnosed cancer). The cancer patient can be treatment naive for one cancer but can be diagnosed with one or more other cancers resulting from, for example, metastasis or malignancy. The cancer patient can be immune checkpoint naive for one or more cancers. The cancer patient can have a cancer that is refractory. In certain instances, the combinations described herein are administered as a first line therapy (e.g., the first therapy administered to a treatment naive cancer patient) to a patient in need thereof.

In some embodiments, the method of treating cancer inhibits metastasis of the cancer in the patient. In some embodiments, metastasis is inhibited by at least about 5%, 10%, 20%, 30%, 40%, 50%, 60%, 70%, 80%, 90%, or 100%. In some other embodiments, the method of treating cancer inhibits brain metastasis.

In some embodiments, the method of treating cancer reduces pre-existing tumor metastasis in the patient. In some other embodiments, the method of treating cancer reduces pre-existing brain metastasis in the patient. In some embodiments, preexisting tumor metastasis is reduced by at least about 5%, 10%, 20%, 30%, 40%, 50%, 60%, 70%, 80%, 90%, or 100%.

In some embodiments, the method of treating cancer prolongs or increases the time to disease progression of the cancer in the patient (including progression between advanced stages; e.g., progression from Stage III to Stage IV cancer). In some embodiments, the increase is a comparison between the time to disease progression with and without treatment. In some embodiments, the methods described herein prolong the time to disease progression by at least 1 week, 2 weeks, 3 weeks, 4 weeks, 1 month, 2 months, 3 months, 4 months, 5 months, 6 months, 7 months, 8 months, 9 months, 10 months, 11 months, 1 year, or more, including values therein.

In some embodiments, the method of treating cancer prolongs the survival of the patient. In some embodiments, the method of treating cancer increases progression-free survival of the patient. In some embodiments, the method of treating cancer prolongs the time to disease progression of the cancer in the patient. In some embodiments, the method of treating cancer prolongs the survival of the patient. In some embodiments, the method of treating cancer increases progression-free survival of the patient. In some embodiments, survival is prolonged by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

In some embodiments, the patient is treatment naïve patient is treatment naïve.

In some embodiments, the method comprises administering the compound of Formula I and compound of Formula II as described herein to the patient as a first line therapy. In some embodiments, the method comprises administering the compound of Formula I and compound of Formula II as described herein to the patient as a second, third, fourth, fifth, or sixth line of treatment. In some embodiments, the method comprises administering the compound of Formula I and compound of Formula II as described herein as a second line of treatment. In some embodiments, the method comprises administering the compound of Formula I and compound of Formula II as described herein as a third line of treatment.

In some embodiments, the method comprises administering the compound of Formula I and compound of Formula II as described herein to the patient following treatment with at least one anti-cancer therapy. In some embodiments, the anti-cancer therapy is chemotherapy, radiotherapy, surgery, targeted therapy, immunotherapy, or a combination thereof. In some embodiments, the anti-cancer therapy is chemotherapy. In some embodiments, the anti-cancer therapy is radiotherapy. In some embodiments, the anti-cancer therapy is cancer surgery. In some embodiments, the anti-cancer therapy is tumor resection or excision. In some embodiments, the anti-cancer therapy is immunotherapy.

In some embodiments, the cancer is resistant to at least one anti-cancer agent.

### Methods of Dosing and Treatment Regimens

In some embodiments, the combinations described herein are used in the preparation of medicaments for the treatment of diseases or conditions described herein.

In some embodiments, the combination disclosed herein are administered for prophylactic and/or therapeutic treatments. In some therapeutic applications, the combinations are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, the combinations disclosed herein are administered to a patient susceptible to or otherwise at risk of a particular disease, disorder or condition.

In some embodiments, the method comprises administering the combinations described herein to the patient orally or by intraperitoneal methods (i.p.) or a combination thereof. In some embodiments, the combination is administered orally. In some embodiments, the combination is administered orally. In some embodiments, the combination is administered by i.p. methods. In some embodiments, the combination is administered intravenously (I.V.).

Doses of compound of Formula I and compound of Formula II employed for adult human treatment are typically in the range of 0.01 mg-5000 mg per day or from about 0.01 mg to about 1000 mg per day. In one embodiment, the desired dose is conveniently presented in a single dose or in divided doses.

In certain embodiments, the dose of drug being administered may be temporarily reduced or temporarily suspended for a certain length of time (i.e., a “drug holiday”).

In some embodiments, the method comprises administering the combinations described herein to the patient daily, weekly, or monthly. In some embodiments, the combination is administered daily. In some embodiments, the combination is administered weekly. In some embodiments, the combination is administered bi-weekly. In some embodiments, the combination is administered monthly. In some embodiments, the combination is administered bi-monthly.

The compounds of Formula I and compound of Formula II as described herein can be administered, for example, once a day (QD), twice daily (BID), once a week (QW), twice weekly (BID), three times a week (TIW), or monthly (QM). In some embodiments, the method comprises administering the combinations described herein QD, BID, or TID. In some embodiments, the combination is administered QD. In some embodiments, the combination is administered BID. In some embodiments, the combination is administered TID. In certain instances, the compounds described herein are administered 2 to 3 times a week. In another embodiment, the compounds described herein are administered QD. The compounds can be administered QD for about: 1 day to about 7 days, 1 day to about 14 days, 1 day to about 21 days, 1 day to about 28 days, or daily until disease progression or unacceptable toxicity. The administration of the compounds described herein can, in part, depend upon the tolerance of the patient where greater tolerance can allow greater or more frequent administration.

The term “administered simultaneously”, as used herein, is not specifically restricted and means that the compounds of the present disclosure and the additional active agent are substantially administered at the same time, e.g. as a mixture or in immediate subsequent sequence.

The term “administered sequentially”, as used herein, is not specifically restricted and means that the compounds of the present disclosure and the additional active agent are not administered at the same time but one after the other, or in groups, with a specific time interval between administrations. The time interval may be the same or different between the respective administrations of the compounds of the present disclosure and the additional active agent and may be selected, for example, from the range of 2 minutes to 96 hours, 1 to 7 days or one, two, or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

In some embodiments, the KRAS inhibitor and the SHP2 inhibitor compound as described herein, or a pharmaceutically acceptable salt or solvate thereof are administered concurrently or sequentially. In some embodiments, the SHP2 inhibitor compound described herein and the KRAS inhibitor are administered sequentially. In some embodiments, the SHP2 inhibitor described herein, or a pharmaceutically acceptable salt or solvate thereof (e.g., a compound of Formula IIa), is administered QD, BID, or TID; and the EGFR TK inhibitor is administered QD, BID, or TID. In some other embodiments, the KRAS inhibitor described herein, or a pharmaceutically acceptable salt or solvate thereof (e.g., a compound of Formula Ia), is administered QD, BID, or TID; and the EGFR TK inhibitor is administered QD, BID, or TID.

The combinations described herein can include administration of each therapy (e.g., a compound of Formula Ia and compound of Formula IIa), where the administration is performed simultaneously or sequentially (in either order). In some embodiments, the SHP2 inhibitor compound described herein and the KRAS inhibitor are administered simultaneously (e.g., within at least 1 to 5 min of each other). In other embodiments, the compound of Formula I and the compound of Formula IIa are administered sequentially (e.g., within at least 10 min, 15 min, 30 min, 1 h, 2 h, 5 h, 10 h, 12 h, 1 day, 2 days, 5 days, 7 days, 14 days, or 21 days of each other).

In some embodiments, the SHP2 inhibitor compound described herein is administered concurrently with a KRAS inhibitor compound. In some embodiments, the SHP2 inhibitor compound described herein is administered prior to the KRAS inhibitor compound. In some embodiments, the SHP2 inhibitor compound described herein is administered after the KRAS inhibitor compound.

The combinations described herein can be administered in a regimen. The regimen can be structured to provide therapeutically effective amounts of a SHP2 inhibitor compound described herein and a KRAS inhibitor compound described herein over a predetermined period of time (e.g., an administration time). The regimen can be structured to limit or prevent side-effects or undesired complications of each of the components of the combination described herein. The regimen can be structured in a manner that results in increased effect for both therapies of the combination (e.g., synergy). Regimens useful for treating cancer can include any number of days of administration which can be repeated as necessary. Administration periods can be broken by a rest period that includes no administration of at least one therapy. For example, a regimen can include administration periods that include 2, 3, 5, 7, 10, 15, 21, 28, or more days. These periods can be repeated. For example, a regimen can include a set number of days as previously described where the regimen is repeated 1, 2, 3, 4, 5, 6, 7, 8, 9, 10, 11, 12, 13, or more times.

Regimens can include a rest period of at least 1, 2, 3, 5, 7, 10, or more days, where at least one therapy is no longer administered to a patient. The rest period can be determined by, for example, monitoring the reaction of the patient to the drug or by measuring the efficacy of the treatment. A rest period can be applicable to a single therapy, such that only one therapy of a combination described herein is discontinued in the rest period but the other therapy or therapies are still administered. Rest periods can be applied to all of the therapies administered to the subject such that the subject receives no therapy for a set period of time during the rest period.

Regimens described herein for the treatment of cancer using the combinations described herein can be continued until disease progression or unacceptable toxicity.

### Biomarkers

In another aspect, presented herein is a method of modulating one or more biomarkers over baseline levels prior to treatment in a patient in need thereof, comprising administering to the patient a pharmaceutical composition comprising a KRAS inhibitor compound and a SHP2 inhibitor compound as described herein, or a pharmaceutically acceptable salt or solvate thereof, (e.g., a compound of Formula Ia and compound of Formula IIa).

In some embodiments, the one or more biomarkers in increased or decreased over baseline levels prior to treatment. In some embodiments, the one or more biomarkers is increased over baseline levels. In some embodiments, the one or more biomarkers is decreased over baseline levels.

In some embodiments, the one or more biomarkers is increased by at least 5%, at least 10%, at least 15%, at least 20%, at least 25%, at least 30%, at least 40%, at least 50%, at least 100%, or at least 150%. In some embodiments, the one or more biomarkers is increased by at least 1.5 times, 2 times, 3 times, 4 times, 5 times, 10 times, 15 times, 20 times, or 25 times. In some embodiments, the one or more biomarkers is decreased by at least 5%, at least 10%, at least 15%, at least 20%, at least 25%, at least 30%, at least 40%, at least 50%, at least 100%, or at least 150%. In some embodiments, the one or more biomarkers is decreased by at least 1.5 times, 2 times, 3 times, 4 times, 5 times, 10 times, 15 times, 20 times, or 25 times.

In some embodiments, the expression of Dual-Specificity Phosphatase 6 (DUSP6) can be used as a biomarker indicative of treatment efficiency with the compounds of Formula I and Formula II or a pharmaceutically effective salts thereof.

In some embodiments, the expression of phosphorylated Extracellular signal-Regulated Kinase (pERK) (and/or its ratio with its phosphorylated variant (ERK)) can be used as a biomarker indicative of treatment efficiency with the compounds of Formula I and Formula II or a pharmaceutically effective salt thereof.

### EXAMPLES

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety.

### Example 1

The objective of this Example is to evaluate the in vivo anti-tumor efficacy of AMG510 (positive control KRAS G12C inhibitor), MRTX 849 (positive control KRAS G12C inhibitor) and HBI-2438 (a KRAS G12C inhibitor of the invention) in NCH-H1373-luc ICA, an intra-carotid artery metastatic brain tumor model, in Nu/Nu mice. HBI-2438 (the compound of Formula Ia) was supplied by Huyabio International, LLC. This intra-carotid artery model is well recognized in the art for evaluating the ability of a target compound to treat metastatic brain tumor.

All the procedures related to animal handling, care and the treatment in the study were performed according to the guidelines approved by the Institutional Animal Care and Use Committee (IACUC) of WuXi AppTec, following the guidance of the Association for Assessment and Accreditation of Laboratory Animal Care (AAALAC). At the time of routine monitoring, the animals were checked daily for tumor growth and any effects of treatments on normal behavior such as mobility, food and water consumption (by looking only), body weight gain/loss (body weights were measured twice per week), eye/hair matting and any other abnormal effect as stated in the protocol. Death and observed clinical signs were recorded on the basis of the numbers of animals within each subset.

Methods: The NCI-H1373-luc tumor cells were maintained in medium supplemented with 10% heat inactivated fetal bovine serum at 37° C. in an atmosphere of 5% CO₂ in air. The tumor cells were routinely subcultured twice weekly. The cells growing in an exponential growth phase were harvested and counted for tumor inoculation. Each mouse was inoculated with NCI-H1373-luc tumor cells (0.5×10⁵) in 100 μL of PBS. The animals were randomized and treatments were started when the Bioluminescence signal reached 8*10⁶. After grouping, the bioluminescence measurements were taken once per week with IVIS (Lumina II).

Each mouse was inoculated with NCI-H1373-luc tumor cells (0.5×10⁵) in 100 μL of PBS. The animals were randomized and treatments were started when the Bioluminescence signal reached 8*10⁶. After grouping, the bioluminescence measurements were taken once per week with IVIS (Lumina II).

### (a) Testing Article Formulation Preparation (Table 1)

| Test | Conc. | |
|---|---|---|
| article | (mg/mL) | Formulation^{a} |
| Vehicle | — | 1% DMSO + 10% solutol (HS-15) + 89% saline |
| AMG510 | 3 | Weigh 32.143 mg AMG510 and add 10.500 mL (2% HPMC E- |
| | | 50, 0.5% Tween-80 in 50 mM Sodium Citrate Buffer, pH 4.0), |
| | | stir and sonicate to get a suspension. |
| MRTX | 3 | Weigh 31.850 mg MRTX 849 and add 10.500 mL (10% |
| 849 | | captisol in 50 mM citrate buffer, pH 5), stir and sonicate to get |
| | | a clear solution. |
| HBI- | 10 | Weigh 27.849 mg HBI-2438 and add 0.022 mL DMSO to |
| 2438 | | obtain a clear solution, add 0.218 mL solutol with proper |
| | | vortex; then add 1.936 mL saline, mix well to get a clear |
| | | solution. |
| | 3 | Dilute 0.45 mL 10 mg/mL solution with 1.05 mL vehicle. |
| | 1.5 | Dilute 0.225 mL 10 mg/mL solution with 1.275 mL vehicle |

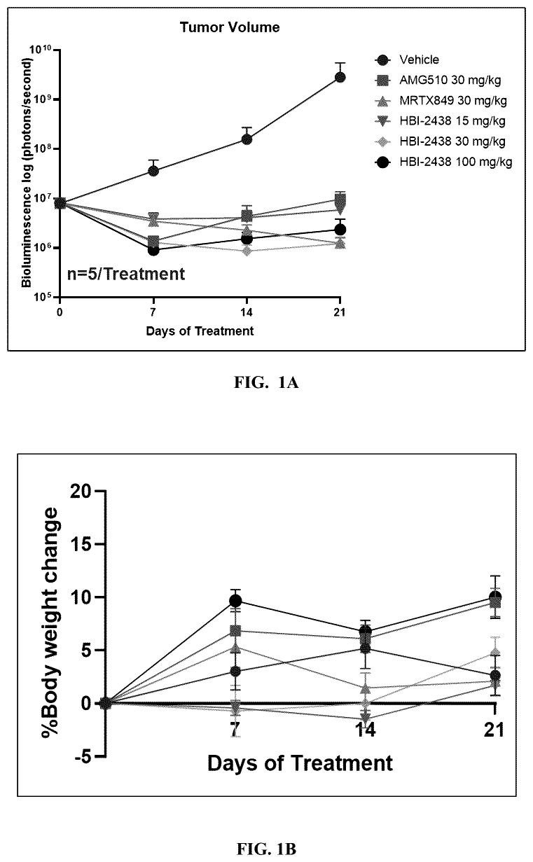

Results: (a) In this study, the therapeutic efficacy of AMG510, MRTX 849 and HBI-2438 in the NCI-H1373-luc ICA tumor model was evaluated. The measured Bioluminescence of all treatment groups at various time points are shown in Table 2 and FIG. 1A.

After 21 days of treatment, the mean log 10 Bioluminescence of vehicle treated mice reached 8.43. Compared with vehicle, AMG-510 at 30 mg/kg PO QD (log 10 Bioluminescence=6.69, TGI=111.5%, T/C=79.3%, p<0.05), MRTX849 at 30 mg/kg PO QD (log 10 Bioluminescence=6.01, TGI=152.6%, T/C=71.3%, p<0.001), HBI-2438 at 15 mg/kg PO QD (log 10 Bioluminescence=6.58, TGI=118.2%, T/C=78.1%, p<0.01), 30 mg/kg PO QD (log 10 Bioluminescence=6.00, TGI=155.8%, T/C=71.2%, p<0.001), and 100 mg/kg PO QD (log 10 Bioluminescence=6.09, TGI=150.0%, T/C=72.2%, p<0.01) all exhibited significant difference. In this study, all mice maintained body weight well during the treatment. Bioluminescence measurement of tumor. See FIG. 1B. Thus, HBI-2438 compared favorably with both the positive controls, thereby demonstrating its utility in treating metastatic tumors, and specifically treatment of metastatic brain tumor.

Mean Bioluminescence of each group at various time point is shown in Table 2.

TABLE 2
Bioluminescence trace over time (mean ± SEM, log 10 Bioluminescence)

| | | Days after the start of treatment | | | |
|---|---|---|---|---|---|
| Group | Treatment | 0 | 7 | 14 | 21 |
| 1 | Vehicle, PO, QD | 6.88 ± 0.07 | 7.27 ± 0.22 | 7.81 ± 0.27 | 8.43 ± 0.45 |
| 2 | AMG-510, 30 mg/kg, PO, QD | 6.86 ± 0.09 | 6.12 ± 0.06 | 6.32 ± 0.25 | 6.69 ± 0.3 |
| 3 | MRTX849, 30 mg/kg, PO, QD | 6.83 ± 0.14 | 6.38 ± 0.21 | 6.24 ± 0.18 | 6.01 ± 0.14 |
| 4 | HBI-2438, 15 mg/kg, PO, QD | 6.86 ± 0.11 | 6.49 ± 0.14 | 6.44 ± 0.2 | 6.58 ± 0.21 |
| 5 | HBI-2438, 30 mg/Kg, PO, QD | 6.86 ± 0.1 | 6.09 ± 0.07 | 5.86 ± 0.12 | 6.00 ± 0.13 |
| 6 | HBI-2438, 100 mg/kg, PO, QD | 6.86 ± 0.11 | 5.92 ± 0.09 | 6.04 ± 0.2 | 6.09 ± 0.24 |

### (b) Tumor Growth Inhibition Analysis and Bioluminescence

Tumor growth inhibition rate for test articles in the NCI-H1373-luc model was calculated based on log 10 Bioluminescence measurement at day 21 after the start of treatment, as shown in Table 3.

TABLE 3
Tumor growth inhibition analysis

| | | | T/C^{b} | TGI^{b} | |
|---|---|---|---|---|---|
| Gr. | Treatment | log 10 Bioluminescence | (%) | (%) | p value ^{c} |
| 1 | Vehicle, PO, QD | 8.43 ± 0.45 | — | — | — |
| 2 | AMG-510, 30 mg/kg, PO, QD | 6.69 ± 0.3 | 79.3 | 111.5 | 0.0119 |
| 3 | MRTX849, 30 mg/kg, PO, QD | 6.01 ± 0.14 | 71.3 | 152.6 | 0.0009 |
| 4 | HBI-2438, 15 mg/kg, PO, QD | 6.58 ± 0.21 | 78.1 | 118.2 | 0.0058 |
| 5 | HBI-2438, 30 mg/Kg, PO, QD | 6.00 ± 0.13 | 71.2 | 155.8 | 0.0008 |
| 6 | HBI-2438, 100 mg/kg, PO, QD | 6.09 ± 0.24 | 72.2 | 150.0 | 0.001 |

a. Mean ± SEM.
^{b}T/C (%) = T/C × 100, TGI (%) = [1 − (Tᵢ − T₀)/(Vᵢ − V₀)] × 100.
^{c} Unpaired two-tailed t test analysis was performed to compare with vehicle group.

### Example 2

The objective of this study was to evaluate preclinically in vivo therapeutic efficacy of test articles HBI-2376 (a SHP2 inhibitor of the invention), HBI-2438 (a KRAS G12C inhibitor of the invention), MRTX849 (positive control KRAS G12C inhibitor) and TNO-155 (positive control SHP2 inhibitor) in the treatment of subcutaneous HuPrime® colorectal cancer xenograft model CR2528 in female BALB/c Nude mice. The above test articles were administered as single agent or in specific combination settings as described in Table 4. The CR2528 model partially responds to treatment with MRTX849, making it a relevant model to test the combination of a KRAS G12C inhibitor (HBI-2438 or MRTX849) and a SHP2 inhibitor (HBI-2376 or TNO-155). HBI-2438 and HBI-2376 (the compound of Formula Ia and the compound of Formula IIa, respectively) were supplied by Huyabio International, LLC.

The report and any amendment(s) or procedures involving the care and use of animals in this study were reviewed and approved by the Institutional Animal Care and Use Committee (IACUC) of CrownBio prior to execution. During the study, the care and use of animals were conducted in accordance with the regulations of the Association for Assessment and Accreditation of Laboratory Animal Care (AAALAC). All the documentations related to this study, including study protocol, protocol amendment, study report, raw data and supporting records were stored in CrownBio for at least 5 years.

Methods: (a) The following dose formulation was used in Example 2 as shown in Table 4:

TABLE 4

| | | Dose | Dosing | | |
|---|---|---|---|---|---|
| | Test | level | Conc. | | |
| Group | article | (mg/kg) | (mg/mL) | Volume | Formulation |
| 1 | Vehicle 1 | — | — | — | 20 g of HP-D-CD was |
| | | | | | dissolved with 100 ml of |
| | | | | | 50 mM sodium citrate |
| | | | | | (pH = 4.2), vortex until |
| | | | | | homogeneous. |
| 2, 8, 9, | HBI-2376 | 5 | 0.5 | 300 μl/mouse × 9 | 45.1 mg of HBI-2376 (37.8 |
| 10 | | | | mice/group × | mg free base, salt factor: |
| | | | | 4 groups × 7 | 1.194) was weighed and |
| | | | | days = 75.6 | dissolved with 75.6 ml |
| | | | | mL | Vehicle 1, vortex to get |
| | | | | | 75.6 ml of 0.5 mg/ml |
| | | | | | dosing solution. Then |
| | | | | | aliquot into 7 vials (10.8 |
| | | | | | ml/vial) for weekly use. |
| 3, 11 | TNO-155 | 5 | 0.5 | 300 μl/mouse × 9 | 18.9 mg of TNO-155 (salt |
| | | | | mice/group × | factor: 1) was weighed and |
| | | | | 2 groups × 7 | dissolved with 37.8 ml |
| | | | | days = 37.8 | Vehicle, vortex to get 37.8 |
| | | | | mL | ml of 0.5 mg/ml dosing |
| | | | | | solution. Then aliquot into 7 |
| | | | | | vials (5.4 ml/vial) for |
| | | | | | weekly use. |
| 1 | Vehicle 2 | — | — | | 1 ml DMSO + 10 ml |
| | | | | | solutol (HS-15) + 89 ml |
| | | | | | Saline (1% DMS0 + |
| | | | | | 10% solutol (HS-15) + |
| | | | | | 89% saline) |
| 5, 8 | HBI-2438 | 10 | 1 | 300 μl/mouse × 9 | 48.40 mg of HBI-2438 |
| | | | | mice/group × | (37.8 mg free base, purity |
| | | | | 2 groups × 7 | 78.1%) was weighed and |
| | | | | days = 37.8 | dissolved with 37.8 ml |
| | | | | mL | Vehicle 2, vortex to get |
| | | | | | 37.8 ml of 1 mg/ml dosing |
| | | | | | solution. Then aliquot into 7 |
| | | | | | vials (5.4 ml/vial) for |
| | | | | | weekly use. |
| 6, 9 | HBI-2438 | 30 | 3 | 300 μl/mouse × 9 | 145.20 mg of HBI-2438 |
| | | | | mice/group × | (113.4 mg free base, purity |
| | | | | 2 groups × 7 | 78.1%) was weighed and |
| | | | | days = 37.8 | dissolved with 37.8 ml |
| | | | | mL | Vehicle 2, vortex to get |
| | | | | | 37.8 ml of 3 mg/ml dosing |
| | | | | | solution. Then aliquot into 7 |
| | | | | | vials (5.4 ml/vial) for |
| | | | | | weekly use. |
| 7, 10 | HBI-2438 | 100 | 10 | 300 μl/mouse × 9 | 483.99 mg of HBI-2438 |
| | | | | mice/group × | (378 mg free base, purity |
| | | | | 2 groups × 7 | 78.1%) was weighed and |
| | | | | days = 37.8 | dissolved with 37.8 ml |
| | | | | mL | Vehicle 2, vortex to get |
| | | | | | 37.8 ml of 10 mg/ml dosing |
| | | | | | solution. Then aliquot into 7 |
| | | | | | vials (5.4 ml/vial) for |
| | | | | | weekly use. |
| | Vehicle 3 | — | — | — | 500 mg CMC was |
| | for | | | | measured into a glass |
| | MRTX849 | | | | beaker, add 100 ml |
| | | | | | injection water and vortex |
| | | | | | until homogeneous. Then |
| | | | | | add 0.5 ml Tween 80 and |
| | | | | | vortex to get a |
| | | | | | homogeneous solution. |
| | | | | | (0.5% CMC + 0.5% |
| | | | | | Tween80) |
| 4, 11 | MRTX849 | 100 | 10 | 300 μl/mouse × 9 | 54 mg of MRTX849 was |
| | | | | mice/group × | weighed and dissolved with |
| | | | | 2 groups = 5.4 | 5.4 ml Vehicle 3. Vortex |
| | | | | mL | and sonicate to get 5.4 ml of |
| | | | | | 3 mg/ml dosing solution. |

(b) The following Model was used:

| | | Cachexia | | |
|---|---|---|---|---|
| Model | Cancer Type | Label | Ulceration Label | Pathology |
| CR2528 | Colorectal | slight | Ulceration | Tubular adenocarcinoma |
| | Cancer | BW loss | | of ascending colon, |
| | | | | moderately differentiated, |
| | | | | ulcerative type, tumor |
| | | | | mass: 4.5 cm × 4 cm × |
| | | | | 0.8 cm. Regional LN: |
| | | | | mesenteric LN (0/20). |

(c) Tumor Inoculation: Tumor fragments from stock mice were harvested and used for inoculation into mice. Each mouse was inoculated subcutaneously in the right flank with CR2528 model tumor fragment (2-3 mm in diameter) for tumor development on May 25, 2022.

(d) Randomization: The randomization started when the mean tumor size reached 160 mm³. 99 mice were enrolled in the study. All animals were randomly allocated to 11 study groups, 9 mice in each group. Randomization was performed based on “Matched distribution” method/“Stratified” method (StudyDirector™ software, version 3.1.399.19)/randomized block design. The date of randomization was denoted as day 0 (Jun. 13, 2022).

Observation and Data Collection: After tumor cells inoculation, the animals were checked daily for morbidity and mortality. During routine monitoring, the animals were checked for any effects of tumor growth and treatments on behavior such as mobility, food and water consumption, body weight gain/loss (body weights were measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were measured by using StudyDirector™ software (version 3.1.399.19).

The treatments on both primary and secondary studies were performed for 3 weeks. The primary study was terminated on Day 21 with plasma and tumor samples collection. For secondary analysis tumor samples were collected after the final dose and were transferred in a sterile tube, to be frozen into liquid nitrogen. All the frozen tumor samples were stored in −80° C.

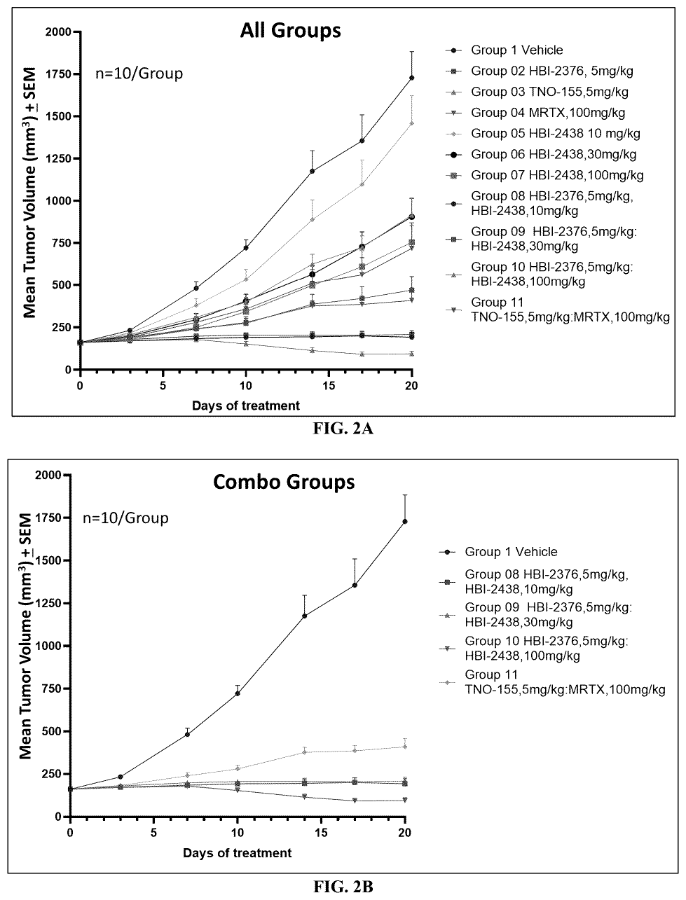

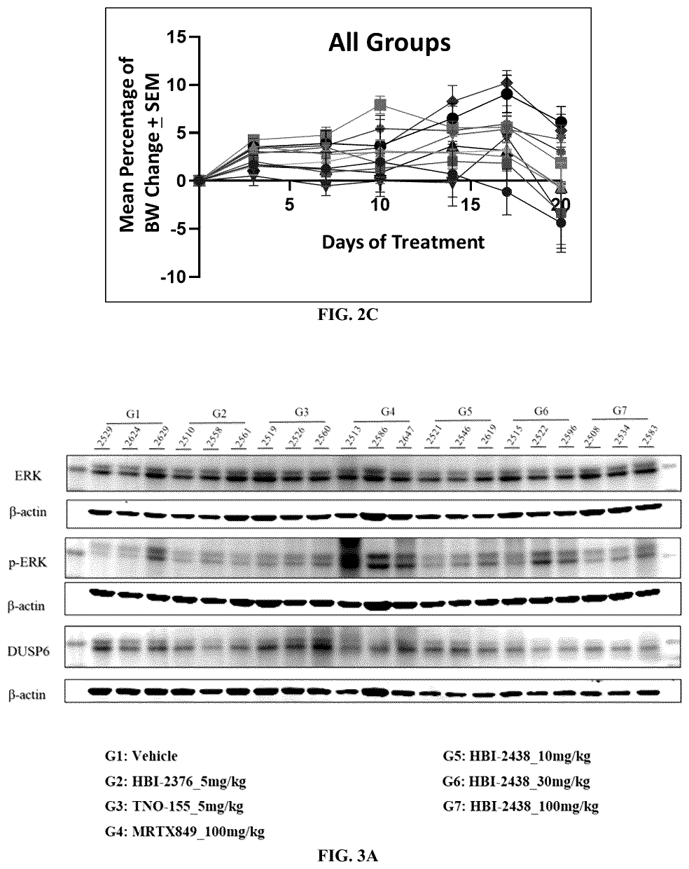

Results: (a) In this study, the therapeutic efficacy of test articles HBI-2376, HBI-2438, TNO-155 and MRTX849 as single agents and in combinations in the treatment of subcutaneous HuPrime® colorectal cancer xenograft model CR2528 in female BALB/c Nude mice was evaluated. Tumor growth inhibition rate for the CR2528 model was calculated at day 21 after the start of treatment, as shown in Table 5 and the tumor volume growth curves between randomization grouping and study termination is shown in FIG. 2A. FIG. 2B is a subset of FIG. 2A, wherein the tumor growth inhibition for the combination treatment with HBI-2376 and HBI-2438 is highlighted.

TABLE 5
Tumor Growth Inhibition Analysis. TGI and T/C calculation on Day 20

| | | Dose | Day 20 | | | |
|---|---|---|---|---|---|---|
| | | Level | Tumor Volume | | | |
| Group | Treatment | (mg/kg) | (mean ± SEM) | T/C (%) | TGI (%) | P Value |
| 1 | Vehicle 1 | — | 1728.03 ± 155.66 | | | |
| | Vehicle 2 | — | | | | |
| 2 | HBI-2376 | 5 | 469.69 ± 81.71 | 27.18 | 72.82 | <0.001 |
| 3 | TNO-155 | 5 | 916.21 ± 100.32 | 53.02 | 46.98 | <0.01 |
| 4 | MRTX849 | 100 | 718.22 ± 151.89 | 41.56 | 58.44 | <0.001 |
| 5 | HBI-2438 | 10 | 1458.01 ± 164.19 | 84.37 | 15.63 | 0.985 |
| 6 | HBI-2438 | 30 | 905.6 ± 108.46 | 52.41 | 47.59 | <0.01 |
| 7 | HBI-2438 | 100 | 754.97 ± 98 | 43.69 | 56.31 | <0.001 |
| 8 | HBI-2376 | 5 | 192.31 ± 28.87 | 11.13 | 88.87 | 0 |
| | HBI-2438 | 10 | | | | |
| 9 | HBI-2376 | 5 | 208.56 ± 22.32 | 12.07 | 87.93 | 0 |
| | HBI-2438 | 30 | | | | |
| 10 | HBI-2376 | 5 | 94.95 ± 15.67 | 5.49 | 94.51 | 0 |
| | HBI-2438 | 100 | | | | |
| 11 | TNO-155 | 5 | 408.66 ± 48.66 | 23.65 | 76.35 | <0.001 |
| | MRTX849 | 100 | | | | |

Note:
Bartlett's test was performed to test homogeneity of variance and normality, P = 1.4e−10, significant, non-parametric test is run to compare groups. P < 0.05 is considered to be statistically significant.

(b) Tolerability and Mortality: Treatments with each compound could be tolerated by test animals. Body weight loss in treated groups was comparable with vehicle control group, probably resulting from the body loss feature of CR2528 model. No other clinical adverse signs were observed during the study period. See FIG. 2C and Table 7.

TABLE 7
Test article related mortality and tolerability.

| | | Dose | Day 0 BW | Day 20 BW | BW | Maximum |
|---|---|---|---|---|---|---|
| | | Level | (mean ± | (mean ± | change | mean BWL |
| Group | Treatment | (mg/kg) | SEM) | SEM) | (%) | (%) |
| 1 | Vehicle 1 | — | 22.29 ± 0.83 | 21.39 ± 1.2 | −4.40% | −4.40% on |
| | Vehicle 2 | — | | | | Day 20 |
| 2 | HBI-2376 | 5 | 22.79 ± 0.69 | 22.03 ± 1.05 | −3.36% | −3.36% on |
| | | | | | | Day 20 |
| 3 | TNO-155 | 5 | 21.68 ± 0.51 | 21.54 ± 0.79 | −0.71% | −0.71% on |
| | | | | | | Day 20 |
| 4 | MRTX849 | 100 | 24.01 ± 0.52 | 25.03 ± 0.62 | 4.30% | No BWL |
| | | | | | | observed |
| 5 | HBI-2438 | 10 | 22.32 ± 0.57 | 22.19 ± 0.81 | −0.62% | −0.62% on |
| | | | | | | Day 20 |
| 6 | HBI-2438 | 30 | 22.54 ± 0.61 | 23.89 ± 0.58 | 6.12% | No BWL |
| | | | | | | observed |
| 7 | HBI-2438 | 100 | 21.91 ± 0.57 | 22.31 ± 0.71 | 1.86% | No BWL |
| | | | | | | observed |
| 8 | HBI-2376 | 5 | 22.02 ± 0.59 | 21.93 ± 1.01 | −0.68% | −0.68% on |
| | HBI-2438 | 10 | | | | Day 20 |
| 9 | HBI-2376 | 5 | 21.73 ± 0.48 | 20.97 ± 0.99 | −3.71% | −3.71% on |
| | HBI-2438 | 30 | | | | Day 20 |
| 10 | HBI-2376 | 5 | 21.56 ± 0.41 | 22.68 ± 0.55 | 5.22% | No BWL |
| | HBI-2438 | 100 | | | | observed |
| 11 | TNO-155 | 5 | 22.97 ± 0.49 | 23.67 ± 0.65 | 3.00% | No BWL |
| | MRTX849 | 100 | | | | observed |

Monotherapy with HBI-2376 at 5 mg/kg QD (Group 02), TNO-155 at 5 mg/kg QD (Group 03), MRTX849 at 100 mg/kg (Group 04), HBI-2438 at 30 mg/kg QD (Group 06) and 100 mg/kg QD (Group 07) suppressed the tumor growth significantly, with TGI value of 72.82% (P<0.001), 46.98% (P<0.01), 58.44% (P<0.001), 47.59% (P<0.01) and 56.31% (P<0.001) respectively. However, after combining HBI-2376 (5 mg/kg, QD) with HBI-2438 at 10 mg/kg QD (Group 08), 30 mg/kg QD (Group 09), and 100 mg/kg QD (Group 10), tumor growth with great retardation was recorded, implying synergistic behavior in the presence of HBI-2376 and HBI-2438. The TGIs were 88.38%, 87.93% and 94.51 (all P=0) respectively. Combining TNO-155 (5 mg/kg QD) with MRTX849 (100 mg/kg QD) (Group 11) generated obvious anti-tumor efficacy, with TGI value of 76.35% (P<0.001). See FIGS. 2A-2C.

### Example 3

### Immunoblot and Gene Expression Analysis

The objective of this study is to evaluate protein/gene expression of pERK, ERK and DUSP6 by western blot and DUSP6 gene expression in the presence of test articles HBI-2376, HBI-2438, and TNO-155 in HuPrime® colorectal cancer xenograft model CR2528. The rationale for this study is that inhibition of phosphorylation and inhibition of DUSP6 are independent indicators of reduced metastaticity.

Method: The treatment was performed for 3 weeks as indicated in Example 2. The fresh tumors were collected in 4 hours after the final dose. Tumor were transferred in sterile tube and thrown into liquid nitrogen for frozen at once. All the frozen tumors were stored in −80° C. Add result summary. Sample for gene expression analysis was also collected.

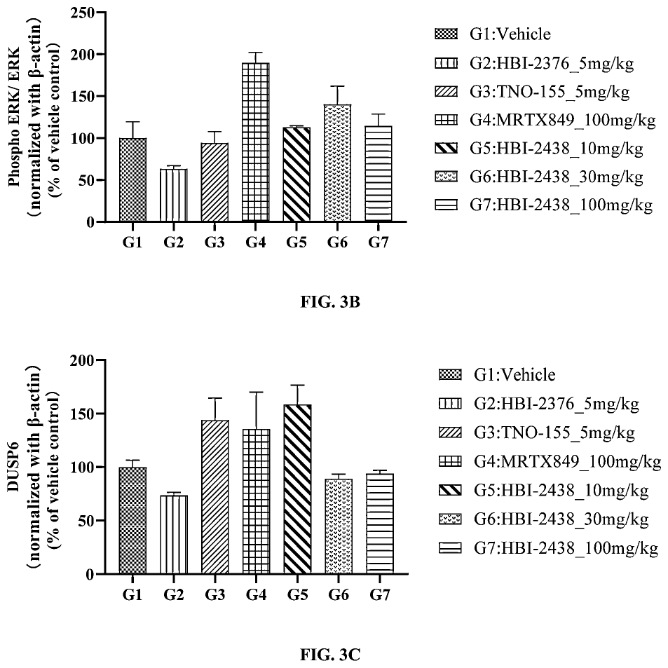

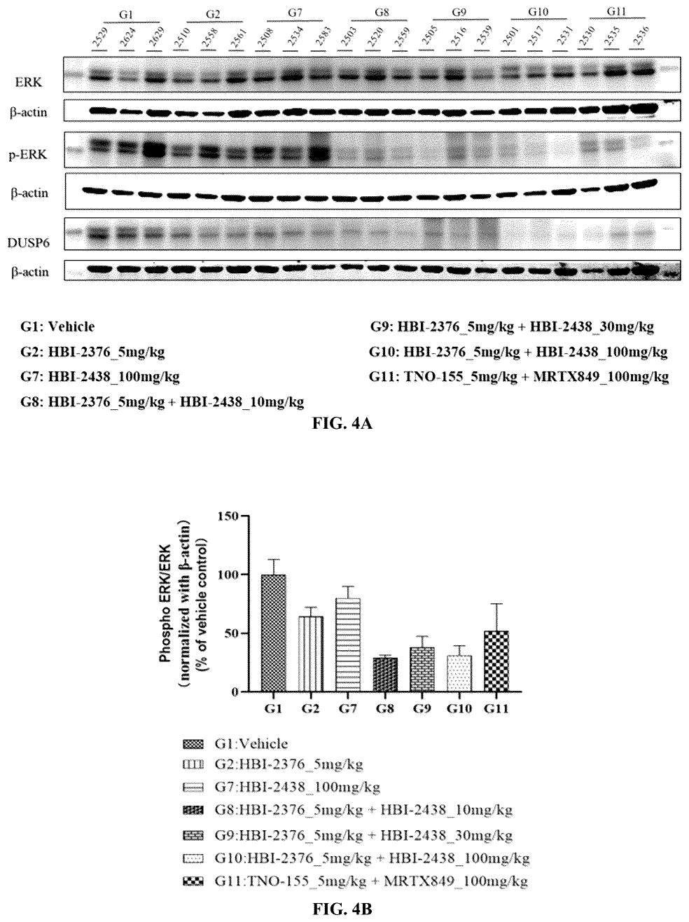

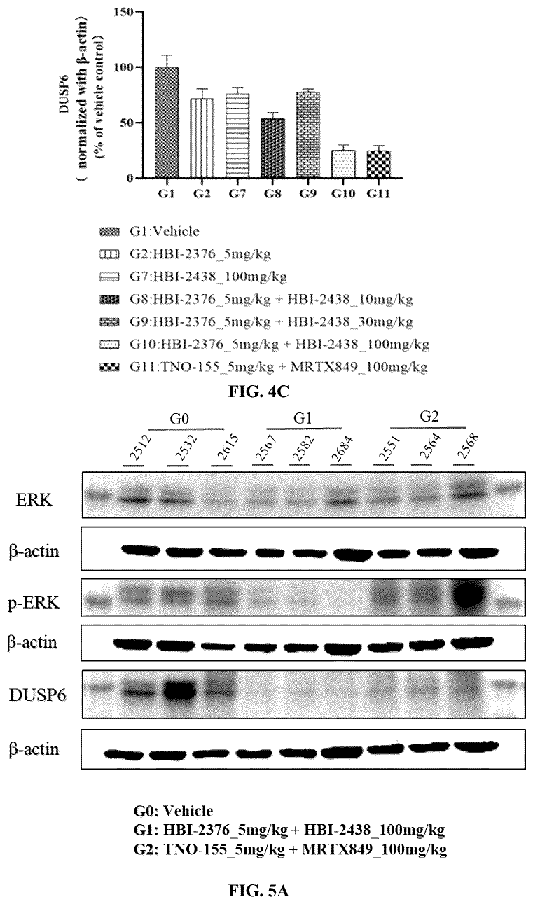

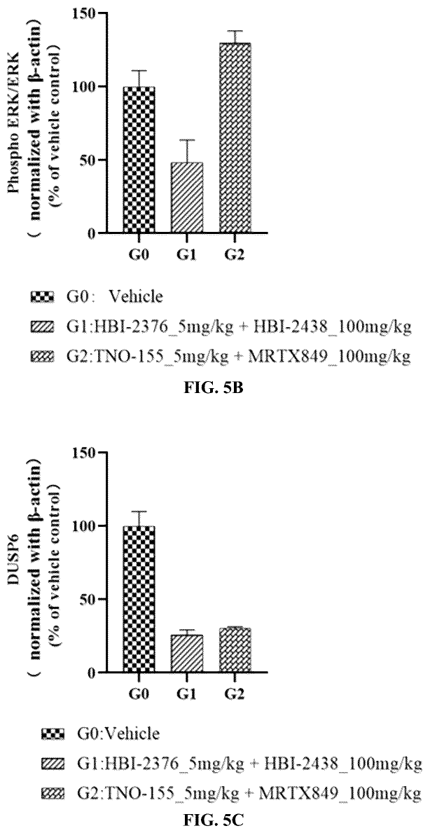

(a) Western blot analysis: Tumors were harvested at the respective time point post dose and were snap frozen in liquid nitrogen. Tissue was ground in liquid nitrogen using a mortar and pestle and weighed. The volume of RIPA buffer containing phosphatase and protease inhibitors was added at 3 times the weight, samples were inverted and placed on ice for 30 min. Cell lysate was obtained by centrifugation at 14,000 g for 15 min at 4° C. and the supernatant was transferred to a fresh tube. Protein was quantified using Pierce BCA Protein Assay kit. 50 μg of protein was loaded in each well. Gels were transferred to preactivated PVDF and primary and secondary antibodies were diluted in TBST with 5% dry milk. Target proteins were detected with Tanon 5200 chemiluminescence image analysis system using ECL method. Primary antibodies: DUSP6, Abcam ab76310, β-actin CST 3700S, p-ERK CST 4370s, ERK CST 4695s. The expression of ERK, pERK, DUSP6 in different treatment groups is shown in FIGS. 3A, 4A and 5A. The ratio of pERK/ERK is measured and relative expression is quantified. See FIGS. 3B, 4B, and 5B. The DUSP6/β-actin expression level is quantified in FIGS. 3C, 4C, and 5C.

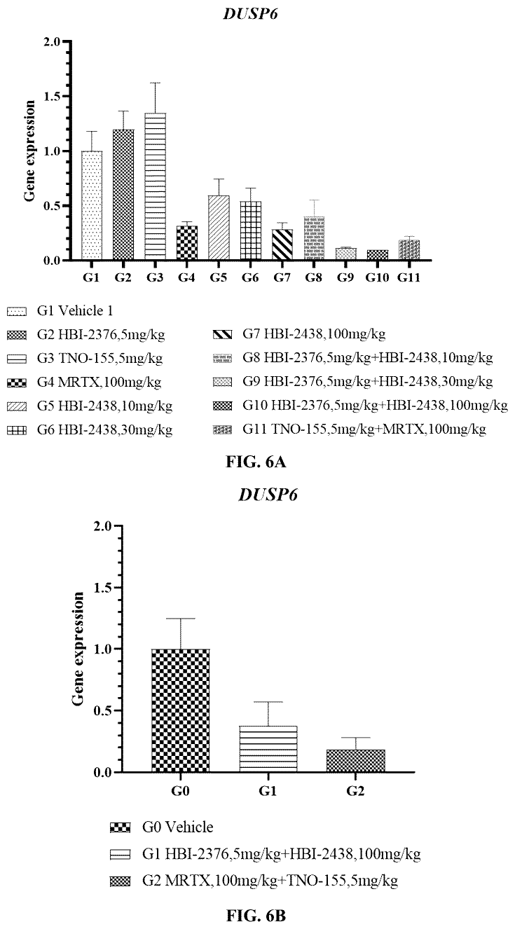

(b) Gene expression analysis: Tumor tissue was harvested 4 hours post dose and tissue was snap frozen in liquid nitrogen. Tissue was ground in liquid nitrogen using a mortar and pestle and weighed. Tissue was placed in RLT buffer containing a stainless steel bead and placed in the TissueLyser. RNA was processed using RNAeasy mini spin kit (Qiagen 74106). Total RNA was quantified by Nanodrop™ 2000 spectrophotometer. cDNA was prepared using High Capacity cDNA Reverse Transcriptase Kit (ABI 4374966). Real time PCR was preformed using TaqMan Universal PCR Master Mix (ABI 4304437) with DUSP6 probe (Theremo Fisher 4331182) and GAPDH probe (ThermoFisher 4351370) using Applied Biosystems Inc, fact PCR system 7900H. Raw data will be analyzed by SDS 2.4 and processed using the ΔCt relative quantification method. ΔCt values will be calculated against the human house-keeping gene, GAPDH. ΔΔCt values will be calculated against the vehicle groups. 2^{-ΔΔCt} represents target gene, DUSP6, expression level. The expression of DUSP6 was thus quantified for different treatment groups. See FIGS. 6A and 6B.

It is to be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating or preventing brain metastasis, comprising administering to the subject having a primary tumor an effective amount of a compound of Formula I, wherein the compound of Formula I is selected from: 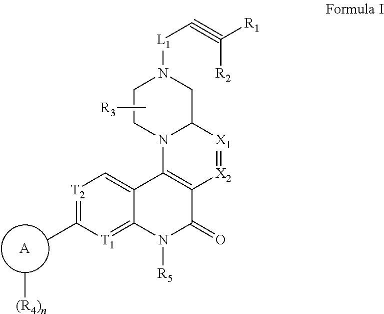
or a pharmaceutically acceptable salt thereof, wherein
R₁, R₂ are independently selected from H, halogen and C₁₋₆ alkyl, the C₁₋₆ alkyl is optionally substituted by 1, 2 or 3 R;
R₃ is selected from H, halogen, OH, NH₂, CN, C₁₋₆ alkyl, C₁₋₆ heteroalkyl, 3-6 membered heterocycloalkyl, C₃₋₆ cycloalkyl, 3-6 membered heterocycloalkyl-O— and C₃₋₆ cycloalkyl-O—, the C₁₋₆ alkyl, C₁₋₆ heteroalkyl, 3-6 membered heterocycloalkyl, C₃₋₆ cycloalkyl, 3-6 membered heterocycloalkyl-O— or C₃₋₆ cycloalkyl-O— is optionally substituted by 1, 2 or 3 R;
R₄ is independently selected from H, halogen, OH, NH₂, CN, C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl and 5-6 membered heteroaryl-fused 5-6 membered heterocycloalkyl, the C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
R₅ is selected from H, C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl and 5-6 membered heteroaryl-fused 5-6 membered heterocycloalkyl, the C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
L₁ is selected from —C(═O)—, —S(═O)— and —S(═O)₂—;
R₆ is selected from H, CN, C₁₋₆ alkyl, C₁₋₆ alkyl-S(═O)₂—, 3-6 membered heterocycloalkyl, —C₁₋₆ alkyl-3-6 membered heterocycloalkyl and C₃₋₆ cycloalkyl-C(═O)—, the C₁₋₆ alkyl, C₁₋₆ alkyl-S(═O)₂—, 3-6 membered heterocycloalkyl, —C₁₋₆ alkyl-3-6 membered heterocycloalkyl or C₃₋₆ cycloalkyl-C(═O)— is optionally substituted by 1, 2 or 3 R;
R₇ is independently selected from H, halogen, OH, NH₂, CN, —C(═O)OH, C₁₋₆ alkyl-O—C(═O)—, —C(═O)—NH₂, C₁₋₆ alkyl, C₁₋₆ heteroalkyl and —C₁₋₆ alkyl-3-6 membered heterocycloalkyl, the C₁₋₆ alkyl, C₁₋₆ heteroalkyl, C₁₋₆ alkyl-O—C(═O)— or —C₁₋₆ alkyl-3-6 membered heterocycloalkyl is optionally substituted by 1, 2 or 3 R;
T₁, T₂ are independently selected from N and —C(R₈)—;
R₈ is selected from H, halogen, OH, NH₂, CN, C₁₋₆ alkyl, C₁₋₆ heteroalkyl, C₃₋₆ cycloalkyl and 3-6 membered heterocycloalkyl, the C₁₋₆ alkyl, C₁₋₆ heteroalkyl, C₃₋₆ cycloalkyl or 3-6 membered heterocycloalkyl is optionally substituted by 1, 2 or 3 R;
R is independently selected from H, halogen, OH, NH₂, CN, 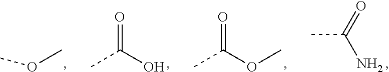
C₁₋₆ alkyl, C₁₋₆ heterocycloalkyl, C₃₋₆ cycloalkyl, 5-6 membered heterocycloalkyl, C₃₋₆ cycloalkyl-O— and 5-6 membered heterocycloalkyl-O—, the C₁₋₆ alkyl, C₁₋₆ heterocycloalkyl, C₃₋₆ cycloalkyl, 5-6 membered heterocycloalkyl, C₃₋₆ cycloalkyl-O— or 5-6 membered heterocycloalkyl-O— is optionally substituted by 1, 2 or 3 R′;
R′ is selected from F, Cl, Br, I, OH, NH₂ and CH₃;
ring A is independently selected from C₆₋₁₀ aryl, 5-10 membered heteroaryl, benzo 5-6 membered heterocycloalkyl and 5-6 membered heteroaryl-fused 5-6 membered heterocycloalkyl;
n is selected from 0, 1, 2, 3 or 4; [symbol]
is [symbol] or [symbol] , and when [symbol] is [symbol] , R₂ is not existed; [symbol]
is [symbol] or;
when in [symbol] , [symbol] is [symbol] , X₁, X₂ are independently selected from —N═, —C(R₇)═ and —C(R₇)₂—C(R₇)═;
when in [symbol] , [symbol] is [symbol] , X₁, X₂ are independently selected from single bond, —O—, —S—, S(═O), S(═O)₂, —N(R₆)—, —C(═O)—, —C(R₇)₂— and —C(R₇)₂—C(R₇)₂—;
the above 3-6 membered heterocycloalkyl, 5-6 membered heteroaryl, 5-6 membered heterocycloalkyl, 5-10 membered heteroaryl or C₁₋₆ heterocycloalkyl comprises 1, 2, or 3 heteroatoms or heteroatomic groups independently selected from —O—, —NH—, —S—, —C(═O)—, —C(═O)O—, —S(═O)—, —S(═O)₂— and N.

2. The method of claim 1, wherein treating brain metastasis comprises suppression of growth of a brain tumor and the effective amount of the compound is an amount effective to suppress growth of a brain tumor.

3. The method of claim 2, wherein the brain tumor is a secondary tumor.

4. The method of claim 3, wherein the primary tumor is not a brain tumor.

5. The method of any one of claim 1, wherein the compound of Formula I penetrates the blood-brain barrier (BBB).

6. The method of any one of claim 1, wherein the compound of Formula I is the compound of formula Ia: 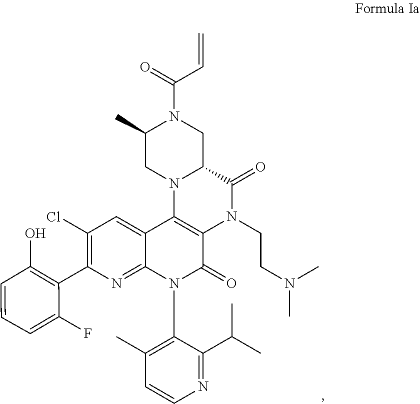 [SMILES: C=CC(=O)N1C[C@@H]2C(=O)N(CCN(C)C)c3c(c4cc(Cl)c(-c5c(O)cccc5F)nc4n(-c4c(C)ccnc4C(C)C)c3=O)N2C[C@H]1C]
or an enantiomer or a pharmaceutically acceptable salt thereof.

7. The method of any one of claim 1, wherein the compound of Formula I or Formula Ia is administered to the subject in an amount of about 1 to about 100 mg/kg per day for at least one day.

8. The method of any one of claim 1, further comprising administering an effective amount a compound of Formula II, or a pharmaceutically acceptable salt or enantiomer thereof, wherein the compound of Formula II is: 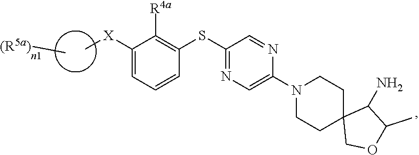
Formula II
wherein:
X is selected from chemical bond, —NH—, —CONH—;
R^{4a} is selected from H, D, halogen atom, —CN, —C(O)OH, —CHO, —OH, —NO₂, —C(O)NHR^{14a} or —NHC(O)R^{15a}, substituted or unsubstituted with the group selected from —NH₂, C₁-C₁₀ alkyl, C₁-C₁₀ alkylamino, C₁-C₁₀ alkoxy, C₃-C₁₂ cycloalkyl, 3-12 membered heterocyclic group, C₆-C₁₀ aryl, or 5-10 membered heteroaryl; wherein R^{14a} and R^{15a} are each independently selected from C₁-C₁₀ alkylamino, C₃-C₁₂ cycloalkyl, C₆-C₁₀ aryl or 5-10 membered heteroaryl group; the substituent is selected from C₁-C₁₀ alkyl, halogen, —NH₂, —CN, —C(O)OH, —CHO, —OH, —NO₂, or C₁-C₁₀ alkoxy, substituted by one or more substituents of C₁-C₁₀ alkylamino, C₃-C₁₂ cycloalkyl, C₆-C₁₀ aryl, 5-10 membered heteroaryl, or 3-12 membered heterocyclic group, the substituents are optionally selected from C₁-C₁₀ alkyl, halogen, —NH₂, —CN, —C(O)OH, —CHO, —OH, —NO₂, C₁-C₁₀ alkoxy, C₁-C₁₀ alkylamino, or C₃-C₁₂ cycloalkyl; 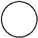
is selected from C₆-C₁₀ aryl, 5-10 membered heteroaryl, C₄-C₁₂ cycloalkyl, 3-12 membered heterocyclic group, C₆-C₁₄ bridged ring group or spiro ring group, or C₆-C₁₄ bridged heterocyclic group or spiro heterocyclic group; wherein the 5-10 membered heteroaryl group, 3-12 membered heterocyclic group, C₆-C₁₄ bridged heterocyclic group or spiro heterocyclic group contains one to three heteroatom or groups selected from N, NH, O, S, C(O), or S(O);
each R^{5a} is the same or different, and is independently selected from H, D, halogen atom, —CN, —C(O)OH, —CHO, —OH, —NO₂, or aminoacyl, substituted or unsubstituted with the group selected from C₁-C₁₀ alkyl, C₁-C₁₀ alkylamino, C₁-C₁₀ alkoxy, —NH₂, C₃-C₁₂ cycloalkyl, 3-12 membered heterocyclic group, C₆-C₁₀ aryl or 5-10 membered heteroaryl, the substituent selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaromatic group, C₆-C₁₀ aryl or 3-12 membered heterocyclic group substituted by one or more substituents; or any two adjacent R^{5a} form a 3-6 membered saturated or unsaturated ring, and is optionally, the 3-6 membered saturated or unsaturated ring is comprises one to three —OH, —NH₂, —CN, halogen, C₁-C₁₀ alkyl, C₁-C₁₀ alkoxy, C₃-C₁₂ cycloalkylamino, C₁-C₁₀ alkylamino, C₃-C₁₂ cycloalkyl, halogenated C₁-C₁₀ alkylamino, C₆-C₁₀ aryl or 5-10 member heteroaryl; and
n1 is 0, 1, 2 or 3.

9. The method of claim 8, wherein the amount of the compound of Formula II is synergistic with the amount of the compound of Formula I.

10. The method of claim 8, wherein the combined amounts of the compound of Formula I and Formula II are effective to suppress growth or induce regression of a brain tumor.

11. The method of claim 10, wherein the combined amounts of the compounds of Formula I and Formula II are effective to induce regression of a brain tumor.

12. The method of claim 10, wherein the brain tumor is a secondary tumor.

13. The method of claim 12, wherein the primary tumor is not a brain tumor.

14. The method of claim 10, wherein the compound of Formula I penetrates the blood-brain barrier (BBB).

15. The method of claim 10, wherein the compound of Formula I is administered to the subject in an amount of about 1 to about 100 mg/kg per day for at least one day.

16. The method of claim 10, wherein the compound of Formula II is the compound of Formula IIa, or a racemate or pharmaceutically acceptable salt thereof, wherein Formula II is:

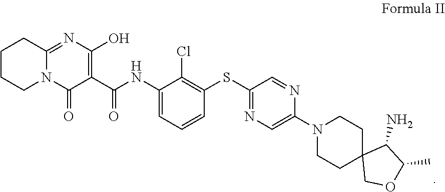 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

17. The method of claim 16, wherein the compound Formula IIa or a pharmaceutically acceptable salt thereof, is administered to said patient in need from about 5 mg/kg to about 25 mg/kg.

18. The method of claim 10, wherein the compound of Formula I is administered to the subject in an amount of about to about 100 mg/kg per day for at least one day.

19. A method of treating cancer, comprising administering to the subject having a tumor an effective amount of a combination comprising an amount of a Kirsten Rat Sarcoma oncogene homologue G12C (KRAS) inhibitor and an amount of a SH2-containing protein tyrosine phosphatase 2 (SHP2) inhibitor, wherein the KRAS inhibitor is a compound of Formula I, or a pharmaceutically acceptable salt thereof, and the SHP2 inhibitor is a compound of Formula II, or a pharmaceutically acceptable salt thereof,
wherein Formula I is: 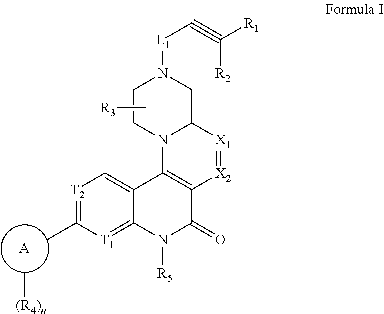
wherein:
R₁, R₂ are independently selected from H, halogen and C₁₋₆ alkyl, the C₁₋₆ alkyl is optionally substituted by 1, 2 or 3 R;
R₃ is selected from H, halogen, OH, NH₂, CN, C₁₋₆ alkyl, C₁₋₆ heteroalkyl, 3-6 membered heterocycloalkyl, C₃₋₆ cycloalkyl, 3-6 membered heterocycloalkyl-O— and C₃₋₆ cycloalkyl-O—, the C₁₋₆ alkyl, C₁₋₆ heteroalkyl, 3-6 membered heterocycloalkyl, C₃₋₆ cycloalkyl, 3-6 membered heterocycloalkyl-O— or C₃₋₆ cycloalkyl-O— is optionally substituted by 1, 2 or 3 R;
R₄ is independently selected from H, halogen, OH, NH₂, CN, C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl and 5-6 membered heteroaryl-fused 5-6 membered heterocycloalkyl, the C₁₋₆ alkyl, C₁₋₆ heteroalkyl, C₃₋₆ cycloalkyl, 3-6 membered heterocycloalkyl, phenyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
R₅ is selected from H, C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl and 5-6 membered heteroaryl-fused 5-6 membered heterocycloalkyl, the C₁₋₆ alkyl, C₃₋₆ cycloalkyl, 5-6 membered heterocycloalkyl-C₁₋₃ alkyl-, 3-8 membered heterocycloalkyl, phenyl, naphthyl, 5-10 membered heteroaryl, benzo 5-6 membered heterocycloalkyl or 5-6 membered heteroaryl-fused 5-6 membered heterocycloalkyl is optionally substituted by 1, 2 or 3 R;
L₁ is selected from —C(═O)—, —S(═O)— and —S(═O)₂—;
R₆ is selected from H, CN, C₁₋₆ alkyl, C₁₋₆ alkyl-S(═O)₂—, 3-6 membered heterocycloalkyl, —C₁₋₆ alkyl-3-6 membered heterocycloalkyl and C₃₋₆ cycloalkyl-C(═O)—, the C₁₋₆ alkyl, C₁₋₆ alkyl-S(═O)₂—, 3-6 membered heterocycloalkyl, —C₁₋₆ alkyl-3-6 membered heterocycloalkyl or C₃₋₆ cycloalkyl-C(═O)— is optionally substituted by 1, 2 or 3 R;
R₇ is independently selected from H, halogen, OH, NH₂, CN, —C(═O)OH, C₁₋₆ alkyl-O—C(═O)—, —C(═O)—NH₂, C₁₋₆ alkyl, C₁₋₆ heteroalkyl and —C₁₋₆ alkyl-3-6 membered heterocycloalkyl, the C₁₋₆ alkyl, C₁₋₆ heteroalkyl, C₁₋₆ alkyl-O—C(═O)— or —C₁₋₆ alkyl-3-6 membered heterocycloalkyl is optionally substituted by 1, 2 or 3 R;
T₁, T₂ are independently selected from N and —C(R₈)—;
R₈ is selected from H, halogen, OH, NH₂, CN, C₁₋₆ alkyl, C₁₋₆ heteroalkyl, C₃₋₆ cycloalkyl and 3-6 membered heterocycloalkyl, the C₁₋₆ alkyl, C₁₋₆ heteroalkyl, C₃₋₆ cycloalkyl or 3-6 membered heterocycloalkyl is optionally substituted by 1, 2 or 3 R;
R is independently selected from H, halogen, OH, NH₂, CN, 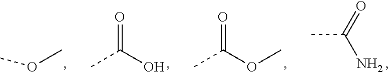
C₁₋₆ alkyl, C₁₋₆ heterocycloalkyl, C₃₋₆ cycloalkyl, 5-6 membered heterocycloalkyl, C₃₋₆ cycloalkyl-O— and 5-6 membered heterocycloalkyl-O—, the C₁₋₆ alkyl, C₁₋₆ heterocycloalkyl, C₃₋₆ cycloalkyl, 5-6 membered heterocycloalkyl, C₃₋₆ cycloalkyl-O— or 5-6 membered heterocycloalkyl-O— is optionally substituted by 1, 2 or 3 R′;
R′ is selected from F, Cl, Br, I, OH, NH₂ and CH₃;
ring A is independently selected from C₆₋₁₀ aryl, 5-10 membered heteroaryl, benzo 5-6 membered heterocycloalkyl and 5-6 membered heteroaryl-fused 5-6 membered heterocycloalkyl;
n is selected from 0, 1, 2, 3 or 4; [symbol]
is [symbol] or [symbol] , and when [symbol] is [symbol] , R₂ is not existed; [symbol]
is [symbol] or;
when in [symbol] , [symbol] is [symbol] , X₁, X₂ are independently selected from —N═, —C(R₇)═ and —C(R₇)₂—C(R₇)═;
when in [symbol] , [symbol] is [symbol] , X₁, X₂ are independently selected from single bond, —O—, —S—, S(═O), S(═O)₂, —N(R₆)—, —C(═O)—, —C(R₇)₂— and —C(R₇)₂—C(R₇)₂—;
the above 3-6 membered heterocycloalkyl, 5-6 membered heteroaryl, 5-6 membered heterocycloalkyl, 5-10 membered heteroaryl or C₁₋₆ heterocycloalkyl comprises 1, 2, or 3 heteroatoms or heteroatomic groups independently selected from —O—, —NH—, —S—, —C(═O)—, —C(═O)O—, —S(═O)—, —S(═O)₂— and N;
and wherein Formula II is: 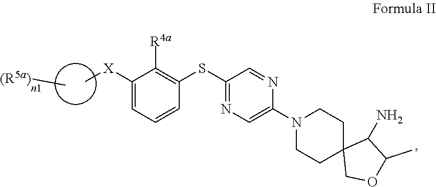
wherein:
X is selected from chemical bond, —NH—, —CONH—;
R^{4a} is selected from H, D, halogen atom, —CN, —C(O)OH, —CHO, —OH, —NO₂, —C(O)NHR^{14a} or —NHC(O)R^{15a} substituted or unsubstituted with the group selected from —NH₂, C₁-C₁₀ alkyl, C₁-C₁₀ alkylamino, C₁-C₁₀ alkoxy, C₃-C₁₂ cycloalkyl, 3-12 membered heterocyclic group, C₆-C₁₀ aryl, or 5-10 membered heteroaryl; wherein R^{14a} and R^{15a} are each independently selected from C₁-C₁₀ alkylamino, C₃-C₁₂ cycloalkyl, C₆-C₁₀ aryl or 5-10 membered heteroaryl group; the substituent is selected from C₁-C₁₀ alkyl, halogen, —NH₂, —CN, —C(O)OH, —CHO, —OH, —NO₂, or C₁-C₁₀ alkoxy, substituted by one or more substituents of C₁-C₁₀ alkylamino, C₃-C₁₂ cycloalkyl, C₆-C₁₀ aryl, 5-10 membered heteroaryl, or 3-12 membered heterocyclic group, the substituents are optionally selected from C₁-C₁₀ alkyl, halogen, —NH₂, —CN, —C(O)OH, —CHO, —OH, —NO₂, C₁-C₁₀ alkoxy, C₁-C₁₀ alkylamino, or C₃-C₁₂ cycloalkyl; 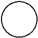
is selected from C₆-C₁₀ aryl, 5-10 membered heteroaryl, C₄-C₁₂ cycloalkyl, 3-12 membered heterocyclic group, C₆-C₁₄ bridged ring group or spiro ring group, or C₆-C₁₄ bridged heterocyclic group or spiro heterocyclic group; wherein the 5-10 membered heteroaryl group, 3-12 membered heterocyclic group, C₆-C₁₄ bridged heterocyclic group or spiro heterocyclic group contains one to three heteroatom or groups selected from N, NH, O, S, C(O), or S(O);
each R^{5a} is the same or different, and is independently selected from H, D, halogen atom, —CN, —C(O)OH, —CHO, —OH, —NO₂, or aminoacyl, substituted or unsubstituted with the group selected from C₁-C₁₀ alkyl, C₁-C₁₀ alkylamino, C₁-C₁₀ alkoxy, —NH₂, C₃-C₁₂ cycloalkyl, 3-12 membered heterocyclic group, C₆-C₁₀ aryl or 5-10 membered heteroaryl, the substituent selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaromatic group, C₆-C₁₀ aryl or 3-12 membered heterocyclic group substituted by one or more substituents; or any two adjacent R^{5a} form a 3-6 membered saturated or unsaturated ring, and is optionally, the 3-6 membered saturated or unsaturated ring is comprises one to three —OH, —NH₂, —CN, halogen, C₁-C₁₀ alkyl, C₁-C₁₀ alkoxy, C₃-C₁₂ cycloalkylamino, C₁-C₁₀ alkylamino, C₃-C₁₂ cycloalkyl, halogenated C₁-C₁₀ alkylamino, C₆-C₁₀ aryl or 5-10 member heteroaryl; and n1 is 0, 1, 2 or 3.

20. The method of claim 19, wherein the method comprises treating metastasis of a tumor.

21. The method of claim 19, wherein treating the tumor comprises treatment of metastasis of the tumor to a brain.

22. The method of claim 21, wherein treatment of metastasis of tumor to the brain comprises suppression of growth of a brain tumor and the effective amount of the compound is an amount effective to suppress growth of the brain tumor.

23. The method of claim 22, wherein the brain tumor is a secondary tumor.

24. The method of claim 23, wherein the primary tumor is not a brain tumor.

25. The method of claim 19, wherein the compound of Formula I penetrates the blood-brain barrier (BBB).

26. The method of claim 19, wherein the compound of Formula I is the compound of Formula Ia: 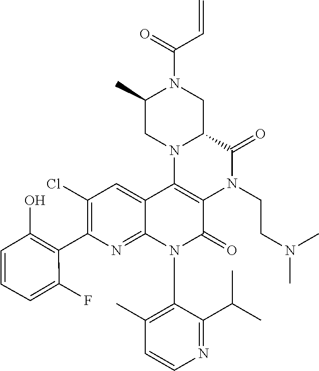 [SMILES: C=CC(=O)N1C[C@@H]2C(=O)N(CCN(C)C)c3c(c4cc(Cl)c(-c5c(O)cccc5F)nc4n(-c4c(C)ccnc4C(C)C)c3=O)N2C[C@H]1C]
Formula Ia, or an enantiomer or a pharmaceutically acceptable salt thereof.

27. The method of claim 19, wherein the compound of Formula I or Formula Ia is administered to the subject in an amount of about 1 to about 100 mg/kg per day for at least one day.

28. The method of claim 27, wherein the amount of the compound of Formula II is synergistic with the amount of the compound of Formula I.

29. The method of claim 27, wherein the combined amounts of the compound of Formula I and Formula II are effective to suppress growth or induce regression of a brain tumor.

30. The method of claim 29, wherein the combined amounts of the compounds of Formula I and Formula II are effective to induce regression of a brain tumor.

31. The method of claim 29, wherein the brain tumor is a secondary tumor.

32. The method of claim 31, wherein the primary tumor is not a brain tumor.

33. The method of claim 27, wherein the compound of Formula I penetrates the blood-brain barrier (BBB).

34. The method of claim 19, wherein the compound of Formula II is the compound of Formula IIa, or a pharmaceutically acceptable salt or racemate thereof, wherein Formula IIa is:

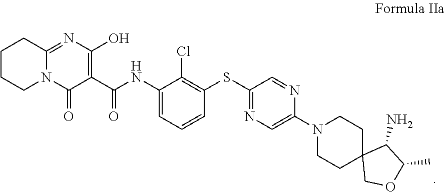 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

35. The method of claim 34, wherein the compound Formula IIa or a pharmaceutically acceptable salt thereof, is administered to said patient in need from about 5 mg/kg to about 25 mg/kg.

36. The method of claim 19, wherein the compound of Formula I is administered to the subject in an amount of about 0.5 to about 100 mg/kg per day for at least one day.
